SEQUENCE LISTING (19 sequences)

SEQ ID NO: 1 - DNA, 272 bp, Homo sapiens
  source 1..272: mol_type = other DNA
gttaacaggcgcgtcccggccaggcggagacgcggccgcggccatgggcgggcgcgggcgcgcggggcggcggtgagggcggctggcggggccgggggcgccgggggggcgcgcgggccgagccgggcctgagccgggcccgcggaccgagctgggagaggggttccggcccccgacgtgctggcgcgggaaaatgttggagatctgcctgaagctggtgggctgcaaatccaagaaggggctgtcctcgtcctccagctgttatctggaag

SEQ ID NO: 2 - RNA, 20 nt, synthetic construct
  source 1..20: mol_type = other RNA
  ncRNA 1..20: ncRNA_class = siRNA
gaaaatgttggagatctgtt

SEQ ID NO: 3 - RNA, 21 nt, synthetic construct
  source 1..21: mol_type = other RNA
  ncRNA 1..21: ncRNA_class = siRNA
gcgggaaaatgttggagattt

SEQ ID NO: 4 - RNA, 21 nt, synthetic construct
  source 1..21: mol_type = other RNA
  ncRNA 1..21: ncRNA_class = siRNA
gggaaaatgttggagatcttt

SEQ ID NO: 5 - RNA, 21 nt, synthetic construct
  source 1..21: mol_type = other RNA
  ncRNA 1..21: ncRNA_class = siRNA
gaagctggtgggctgcaaatt

SEQ ID NO: 6 - RNA, 21 nt, synthetic construct
  source 1..21: mol_type = other RNA
  ncRNA 1..21: ncRNA_class = siRNA
aagctggtgggctgcaaattt

SEQ ID NO: 7 - DNA, 144 bp, Homo sapiens
  source 1..144: mol_type = other DNA
acagtttaacttttgtgcttctgggcagaggtatggtccttgggacagttctccttccacctaatagttatggcagagatcaggacacttcactttgctgcctgtgcactgaggcctcagaatctgctctacccgacttaacag

SEQ ID NO: 8 - RNA, 21 nt, synthetic construct
  source 1..21: mol_type = other RNA
  ncRNA 1..21: ncRNA_class = siRNA
gggcagaggtatggtcctttt

SEQ ID NO: 9 - RNA, 21 nt, synthetic construct
  source 1..21: mol_type = other RNA
  ncRNA 1..21: ncRNA_class = siRNA
acctaatagttatggcagatt

SEQ ID NO: 10 - RNA, 21 nt, synthetic construct
  source 1..21: mol_type = other RNA
  ncRNA 1..21: ncRNA_class = siRNA
gatcaggacacttcacttttt

SEQ ID NO: 11 - RNA, 21 nt, synthetic construct
  source 1..21: mol_type = other RNA
  ncRNA 1..21: ncRNA_class = siRNA
gcagagatcaggacacttctt

SEQ ID NO: 12 - RNA, 21 nt, synthetic construct
  source 1..21: mol_type = other RNA
  ncRNA 1..21: ncRNA_class = siRNA
tggcagagatcaggacacttt

SEQ ID NO: 13 - DNA, 140 bp, Homo sapiens
  source 1..140: mol_type = other DNA
ggcgggcgcaggatgagggcggccattgctggggctccgcttcggggaggaggacgctgaggaggcgccgagccgcgcagcgctgcgggggaggcgcccgcgccgacgcggggcccatggccaggaccaccagccagctg

SEQ ID NO: 14 - RNA, 21 nt, synthetic construct
  source 1..21: mol_type = other RNA
  ncRNA 1..21: ncRNA_class = siRNA
gggaggaggacgctgaggatt

SEQ ID NO: 15 - RNA, 21 nt, synthetic construct
  source 1..21: mol_type = other RNA
ggaggaggacgctgaggagtt

SEQ ID NO: 16 - RNA, 21 nt, synthetic construct
  source 1..21: mol_type = other RNA
  ncRNA 1..21: ncRNA_class = siRNA
aggaggacgctgaggaggctt

SEQ ID NO: 17 - RNA, 21 nt, synthetic construct
  source 1..21: mol_type = other RNA
  ncRNA 1..21: ncRNA_class = siRNA
tcggggaggaggacgctgatt

SEQ ID NO: 18 - RNA, 21 nt, synthetic construct
  source 1..21: mol_type = other RNA
  ncRNA 1..21: ncRNA_class = siRNA
gcttcggggaggaggacgctt

SEQ ID NO: 19 - RNA, 21 nt, synthetic construct
  source 1..21: mol_type = other RNA
  ncRNA 1..21: ncRNA_class = siRNA
ggccaggaccaccagccagtt

(19) United States
(12) Patent Application Publication
(10) Pub. No.: US 2025/0320501 A1
(43) Pub. Date: Oct. 16, 2025

(54) PALMITOYLATION OF THE ALTERNATIVE AMINO TERMINUS OF THE BTK-C ISOFORM CONTROLS SUBCELLULAR DISTRIBUTION AND SIGNALING

(71) Applicant: The Research Foundation for the State University of New York, Albany, NY (US)

(72) Inventors: Douglas Conklin, Rensselaer, NY (US); Mostafa Kokabee, Wakefield, MA (US); Xianhui Wang; Ayesha Tahir, Albany, NY (US)

(21) Appl. No.: 18/869,130

(22) PCT Filed: May 25, 2023
(86) PCT No.: PCT/US23/67484

§ 371 Date: Nov. 25, 2024

Related U.S. Application Data
(60) Provisional application No. 63/345,605, filed on May 25, 2022.

(51) Int. Cl. C12N 15/113

(52) U.S. Cl. CPC C12N 15/1137; C12N 2310/11; C12N 2310/14

(57) ABSTRACT

The BTK-C isoform is expressed in human breast and prostate cancer cells and it plays a crucial role in epithelial cancer cell survival. BTK-C has a 34 amino acid extension to the n-terminus that facilitate it being palmitoylated on two cysteine residues. This modification localizes BTK-C closer to the cell membrane where it plays a role in improving cancer cell survival, as compared to BTK-A. BTK-C is not found to be expressed in healthy adult cells, whereas BTK-A is necessary for immune cell production. Disclosed herein are alternative kinases besides BTK having similar attributes, which present therapeutic opportunities for the treatment of cancers, especially for solid tumors.

### CROSS-REFERENCE TO RELATED APPLICATIONS

This application claims priority to U.S. provisional application No. 63/345,605 filed May 25, 2022, herein entirely incorporated by reference.

### GOVERNMENT SUPPORT CLAUSE

This invention was made with government support under W81XWH-04-1-0474 awarded by the Medical Research and Development Command. The government has certain rights in the invention.

### BACKGROUND OF THE INVENTION

### Field of the Invention

The phosphatidylinositol 3-kinase (PI3K) pathway is commonly activated in a variety of cancers. Increased activity of upstream receptors and mutations in PI3K components lead to production of phosphatidylinositol 3,4,5-triphosphate, PIP₃, on the inner leaflet of the plasma membrane. PIP₃ levels recruit and activate effectors through interaction with pleckstrin homology (PH) domains in a variety of effector proteins. PIP₃ levels are also controlled by the phosphatase and tensin homologue (PTEN). Genomic alterations affect several points in the pathway and are frequently found in most solid tumor types. Loss of PTEN is the most common alteration, however; between a quarter and a half of tumors of various epithelial origins carry mutations in genes in the pathway.

We previously identified an isoform of the PH domain-containing kinase, Bruton's tyrosine kinase (BTK), in an RNAi kinome screen as a critical survival factor for breast cancer cells. ShRNA-mediated knockdown of this BTK isoform (BTK-C; UniProt: Q06187-2, Ensembl: ENST00000621635.4) had a larger effect on reducing survival and proliferation than other targeted kinases with established roles in breast cancer including HER2/neu, EGFR, Src, and others. The BTK-C isoform identified in breast and prostate cancer cells is similar to the original BTK isoform, which we refer to as BTK-A for clarity but contains an alternative first exon encoding parts of a 34 amino acid amino-terminal extension. The BTK-C protein provides cell survival activities in breast and prostate cancer cells whereas BTK-A is expressed in hematopoietic cells during maturation of B-cells. In hematopoietic cells, B-cell receptor engagement by antigen stimulates PI3K and the resulting PIP₃ accumulation at the plasma membrane recruits and activates BTK-A. The importance of BTK-A to B cell survival is underscored by the development BTK inhibitors such as ibrutinib, acalabrutinib and others. These BTK inhibitors also target BTK-C decreasing tumor cell proliferation by reducing resistance to apoptosis which ultimately may be due to effects on glucose transport. Decreased survival signaling caused by BTK-C inhibition also results in decreased therapeutic escape in breast cancer cells suggesting that it may be useful as an adjuvant therapy. Importantly, inhibition of BTK-C prevents activation of the AKT signaling pathway by NRG or EGF that has been shown to promote growth factor-driven lapatinib resistance in HER2+ breast cancer cells. BTK-C signaling is involved in the appearance of ligand-dependent lapatinib resistance in treated HER2-positive breast cancer cell populations. (U.S. Pat. Nos. 8,513,212; 9,095,592; 9,637,554; 10,421,820; and 11,149,092, hereby incorporated by reference)

As discussed herein, the N-terminal extension of the BTK-C isoform contains a which alters its subcellular localization. The palmitoylated form of BTK is expressed in 10-25% of a wide variety of cancers. In breast tumors that do not express BTK-C, increased levels of other signaling inputs are commonly found. In this sense, the BTK-C isoform may function in cancer cells by providing increased effector function in the PI3K pathway. The unique structure of the BTK gene allows for both palmitoylated and non-palmitoylated forms of the kinase to be produced. This appears to be a common feature of mammals, as we find similar exon arrangements in other tyrosine kinases and pleckstrin homology-containing kinase genes. Thus, therapeutic opportunities exist in the exploitation of the palmitoylated isoforms of various tyrosine kinases. The present disclosure provides improved treatments that inhibit cancer cell growth but spare the immune system by either targeting the isoform with small molecule inhibitors or suppressing the translation of the isoform through RNA interference (“RNAi”).

### BRIEF SUMMARY OF THE INVENTION

Embodiments disclosed herein include methods of treating cancer in a subject in need thereof, the method comprises administering an inhibitor of palmitoylation of BTK-C. Embodiments disclosed herein include BTK-C inhibitors that block the palmitoylation of BTK-C at the C residues 13 and 16 indicated in SEQ ID 21. Embodiments disclosed herein include inhibitors selected from the group consisting of nucleic acids, small molecules, peptides, vectors, and antibodies, wherein optionally the nucleic acid is selected from the group consisting of an siRNA, miRNA, an antisense nucleic acid, and an shRNA. Embodiments disclosed herein include methods of treating cancer in a subject in need thereof, the method comprising administering an inhibitor of palmitoylation of a ALB1 kinase. Embodiments disclosed herein include methods of treating cancer in a subject in need thereof, the method comprising administering an inhibitor of palmitoylation of a ALB2 kinase. Embodiments disclosed herein include methods of treating cancer in a subject in need thereof, the method comprising administering an inhibitor of palmitoylation of a PDPK1 kinase. Embodiments disclosed herein include antisense nucleic acids selected from the group consisting of SEQ ID NO. 2, SEQ ID NO. 3, SEQ ID NO. 4, SEQ ID NO. 5, and SEQ ID NO. 6, or antisense nucleic acids selected from the group consisting of SEQ ID NO. 8, SEQ ID NO. 9, SEQ ID NO. 10, SEQ ID NO. 11, and SEQ ID NO. 12, or antisense nucleic acids selected from the group consisting of SEQ ID NO. 14, SEQ ID NO. 15, SEQ ID NO. 16, SEQ ID NO. 17, SEQ ID NO. 18, and SEQ ID NO. 19.

Embodiments disclosed herein include methods of treating cancer in a subject in need thereof, the method comprises administering an inhibitor of palmitoylation, wherein the inhibitor is an siRNA that corresponds to an exon sequence that code for the palmitoylated versions of BTK-C, ABL1, ABL2, and PDPK1. Embodiments disclosed herein include pharmaceutical compositions comprising a means for reducing the amount of palmitoylation sequences in BTK-C, ABL1, ABL2, and PDPK1 kinases in cancerous cells and a pharmaceutically acceptable carrier.

The embodiments of the present disclosure provide improved treatments that inhibit cancer cell growth but spare the immune system. Embodiments disclosed herein include, for example, targeting the palmitoylated isoforms of BTK-C, ABL1, ABL 2, and PDPK1, which are the predominant isoforms expressed in several solid tumor types with either small molecule inhibitors or suppressing the translation of the isoform through RNA interference (“RNAi”).

The present invention does not intend to limit the type of cancer being treated to breast cancer. Cancers that may be treated using the compositions and methods of the present invention include, for example, leukemia, carcinoma, lymphoma, astrocytoma, sarcoma, glioma, retinoblastoma, melanoma, Wilm's tumor, bladder cancer, colon cancer, hepatocellular cancer, pancreatic cancer, prostate cancer, lung cancer, liver cancer, stomach cancer, cervical cancer, testicular cancer, renal cell cancer, and brain cancer.

The present invention does not intend to limit the types of RNA used to silence gene expression via RNA interference (RNAi). According to an embodiment, the present invention contemplates the use of shRNAs, siRNAs, microRNAs (miRNAs), and single- or double-stranded analogues thereof, for silencing gene expression.

The present invention does not intend to limit the compounds and/or molecules used to silence gene expression to dsRNA molecules, such as shRNAs and siRNAs. In one embodiment, the present invention contemplates that inhibitors of cancer cells (e.g. breast cancer) may include small molecule inhibitors.

### REFEFERENCE TO A SEQUENCE LISTING

This application contains a Sequence Listing in an ASCII plain text file, which is incorporated herein by reference. The Sequence file name is 010-22-13WO01_SEQ. The text file was created on May 23, 2023, and the size of the text file is 23,831 bytes.

### BRIEF DESCRIPTION OF THE SEVERAL VIEWS OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

The patent or application file contains at least one drawing executed in color. Copies of this patent or patent application publication with color drawing(s) will be provided by the Office upon request and payment of the necessary fee.

FIG. 1A-D present an overview of the BTK locus in humans: FIG. 1A presents BTK-C transcription is initiated at an alternative transcriptional start site. Encoded sequence in this exon is phylogenetically conserved in mammals; FIG. 1B presents the key feature encoded in the sequence is one or more predicted palmitoylation sites; FIG. 1C presents the BTK-C isoform is expressed in approximately 15% of breast cancer tumors. BTK-C expression is not correlated with sample type; and

FIG. 1D presents Volcano plot of reverse-phase protein array data of BTK-C-expressing and -non-expressing tumors from the TCGA Firehose Legacy dataset. Only those protein species which have statistically significant (p<0.05 and q<0.05) differences in abundance between the two groups are labeled. Human breast tumors that do not express BTK-C are enriched in common breast cancer signaling inputs including ERBB2, the estrogen receptor, androgen receptor and the PIP3-dependent serine threonine kinase, PDPK1.

FIG. 2A-D presents the BTK-C isoform palmitoylated: FIG. 2A presents a diagram of BTK isoform C-terminal Flag-tagged constructs: FIG. 2B presents an expression of Flag-tagged constructs in MDA-MB-231 cells; FIG. 2C presents detection scheme for acylated BTK isoforms. Cells were incubated with 17-ODYA. Flag immunoprecipitates were reacted with azide-activated biotinylation reagent and detected through reaction with streptavidin-linked horseradish peroxidase; and FIG. 2D presents HEK293 cells were transfected with 1 μg of BTK-A, BTK-C, or BTK-C C13A, C16A and 2 μg of a plasmid expressing zDHHC5. 24 h after transfection cells were incubated for 4 h with 100 μM palmitic acid alkyne (17-ODYA). Cell lysate supernatants were immunoprecipitated with Anti-Flag M2 affinity gel (Sigma) at 4° C. overnight, subjected to click chemistry with 20 μM biotin picolyl azide for 30 m at room temperature before electrophoresis and blotting.

FIG. 3A-D presents subcellular distribution of the BTK isoforms in breast cancer cells: FIG. 3A presents a diagram of BTK pleckstrin homology domain with GFP reporters. Expression of each reporter was driven by a CMV promoter in a retroviral vector. FIG. 3B presents localization of wild type and mutant isoform reporters in breast cancer cell lines MD-AMB-231 (triple negative; PTEN+), BT549 (triple negative; PTEN), SUM149 (triple negative; PTEN-) and SKBr3 (luminal HER2 enriched; PTEN+). BTK-A isoform is found in the plasma membrane (red arrows) and to a greater degree in the nuclei of cancer cells (white arrows). BTK-C is also found on the plasma membrane but exhibits a greater degree of perinuclear localization (gray arrows). Mutation of the two palmitoylation sites reduces membrane localization of BTK-C. Mutation of arginine 28 of BTK (arginine 62 and BTK-C sequence) also decreases membrane staining. FIG. 3C presents subcellular fractionation showing increased levels of BTK-A in the nucleus and greater BTK-C in membrane fractions. FIG. 3D BTK reporters localize with actin in cancer cells. Scale bars: 100 μm.

FIG. 4A-D present BTK isoform localization and activation is responsive to PI3K pathway signaling in solid tumor cells: FIG. 4A presents BTK isoform localization in MDA-MB-231 (PTEN+) and SUM149 (PTEN-) cells; FIG. 4B presents pharmacological inhibition of PI3K decreases association of BTK-A and BTK-C with the plasma membrane in LNCaP C4-2b (PTEN-) cells (Scale bar: 100 μm): FIG. 4C presenst activating tyrosine phosphorylation of the BTK isoforms in transiently transfected HEK293 cells. Y551 phosphorylation is dependent on other kinases, Y223 autophosphorylation results from BTK activation. Values represent fold tyrosine-phosphorylated signal normalized to total transfected BTK control (anti-Flag) determined by densitometry; and FIG. 4D presents PIP3 dependence of BTK-C activation in transfected LNCaP C4-2b (PTEN-) cells. PTEN activity by expression of PTEN dominant negative mutants increases BTK-C activation. Additionally, a BTK-C non-kinase domain construct (PH-EGFP) capable of dimerizing with full length BTK reduces activating phosphorylation. In each case, 24 hours after transfection cell lysates were collected for immunoblotting.

FIG. 5A-D present activating tyrosine phosphorylation of the BTK isoforms. BTK-A, BTK-C and BTK-C C13, C16 mutant constructs were transfected into HEK293 cell and after 24 hours stimulated with insulin. Values represent fold tyrosine-phosphorylated signal normalized to total BTK-A or BTK-C(anti-Flag) determined by densitometry; FIG. 5B presents phosphorylation of the BTK target tyrosine 759 of PLCγ2 in transfected HEK293 cells; FIG. 5C presents BTK-C expression increases proliferation rate of MDA-MB-231 (PTEN+) but not SUM149 (PTEN−) cells. Cells were seeded in 96 well plates and fixed with 4% formaldehyde and counted at 72 hours. Data were normalized to control and are presented as mean+SD. *p<0.05 using Student's ttest, n=3; and FIG. 5D presents BTK-C increases glucose uptake in MDA-MB-231 (PTEN+) but not SUM149 (PTEN−) cells. Cells were seeded and treated with 100 μM 2-NBDG for 15 min. Fluorescence images were acquired with an InCell 2200 and overall fluorescence intensity quantified. Data were normalized to control and are presented as mean+SD. *p<0.05 using Student's t-test, n=3.

FIG. 6A-B present alternative N-terminal kinases in the human genome: FIG. 6A presents exon maps of kinases with alternative first exons that either do or do not contain palmitoylation sites, as occurs with BTK-A and BTK-C; and FIG. 6B presents alternate N terminal sequences of tyrosine kinases TXK, FRK, HCK, ABL1, ABL2, and the serine/threonine kinase PDPK1 resemble BTK. Isoforms containing palmitoylation sequences in the amino terminal region are enriched in cysteine and methionine residues in predicted protein products. The encoded alternative isoforms represent a binary switch as non-palmitoylated forms are depleted of cysteine and non-start codon methionine residues near the N terminus.

FIG. 7 presets expression of ABL1 and ABL2 isoforms in a variety of tumor types. Mutation of the non-palmitoylated version of ABL1 is an established driver in acute myeloid leukemia. For this reason, the non-palmitoylated version is known as the canonical version of this kinase. Expression analysis in other tumors shows that for either ABL1 or ABL 2 the palmitoylated version displays higher expression levels.

FIG. 8 presents patients with breast tumors that only express the palmitoylated isoform of ABL2 exhibit decreased overall survival. From TCGA data. P-value generated with Logrank test.

FIG. 9 presents human breast tumors that are in the highest quintile with respect to palmitoylated isoform expression of ABL1, ABL2, and PDPK1 have lower expression of the estrogen receptor.

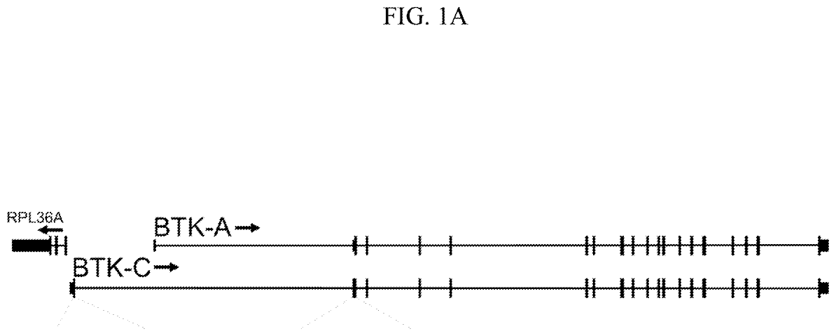

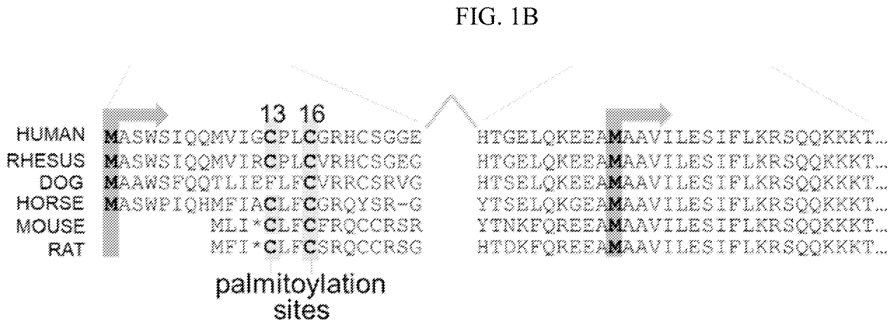

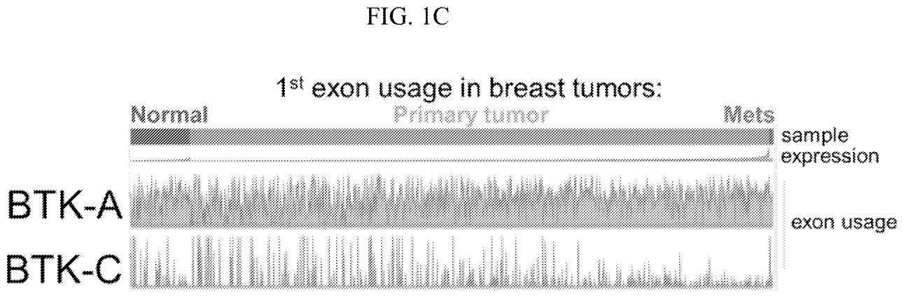

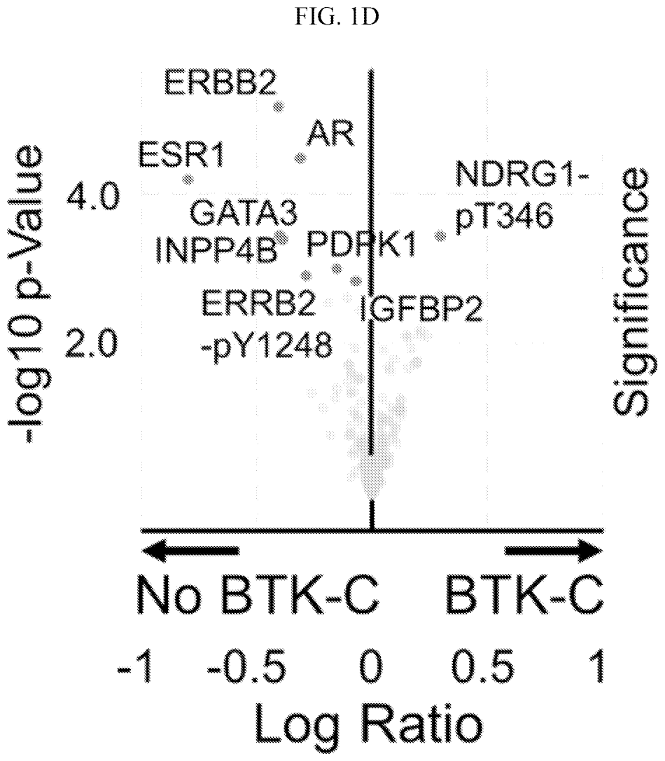

FIG. 1S presents an analysis of recent RNAseq data from the Genotype-Tissue Expression (GTEx) project.

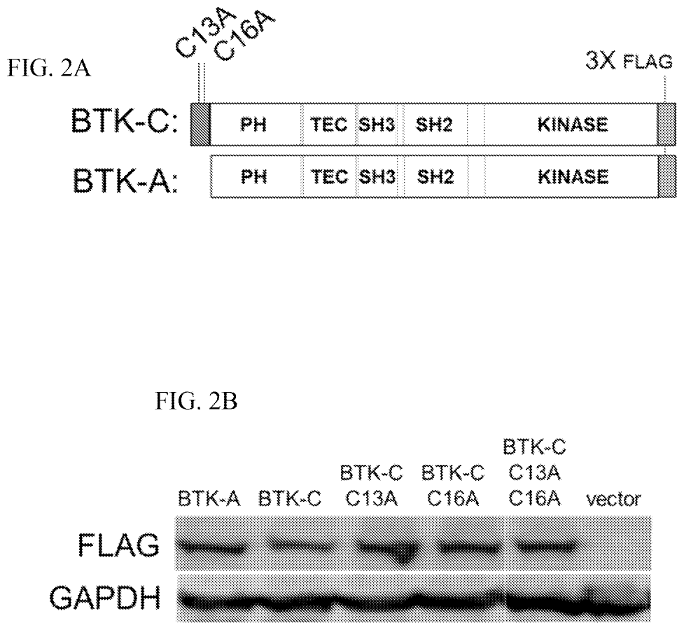

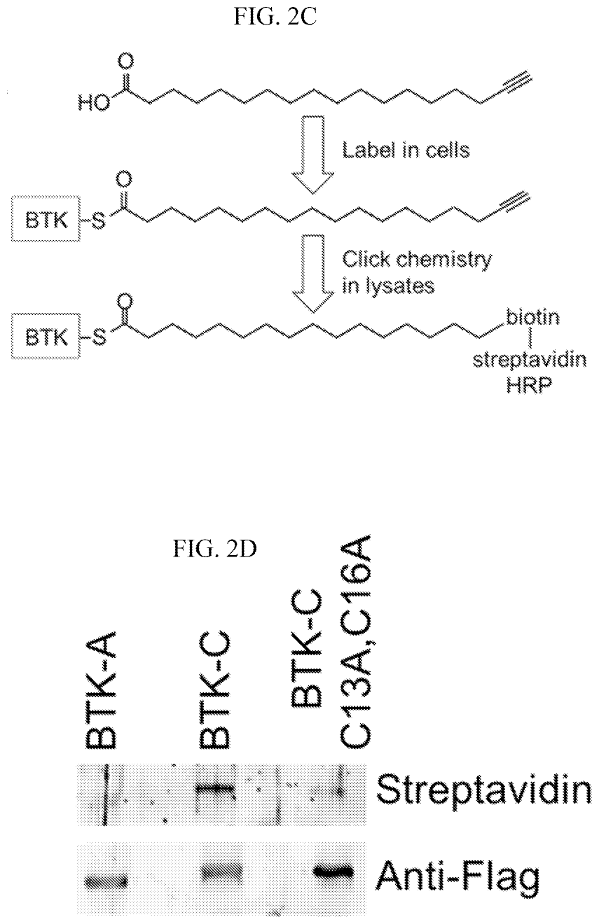

FIG. 2S presents tumor samples from the TCGA Firehose Legacy breast cancer database parsed into two groups: tumors that express BTK-C and those that do not express BTK-C.

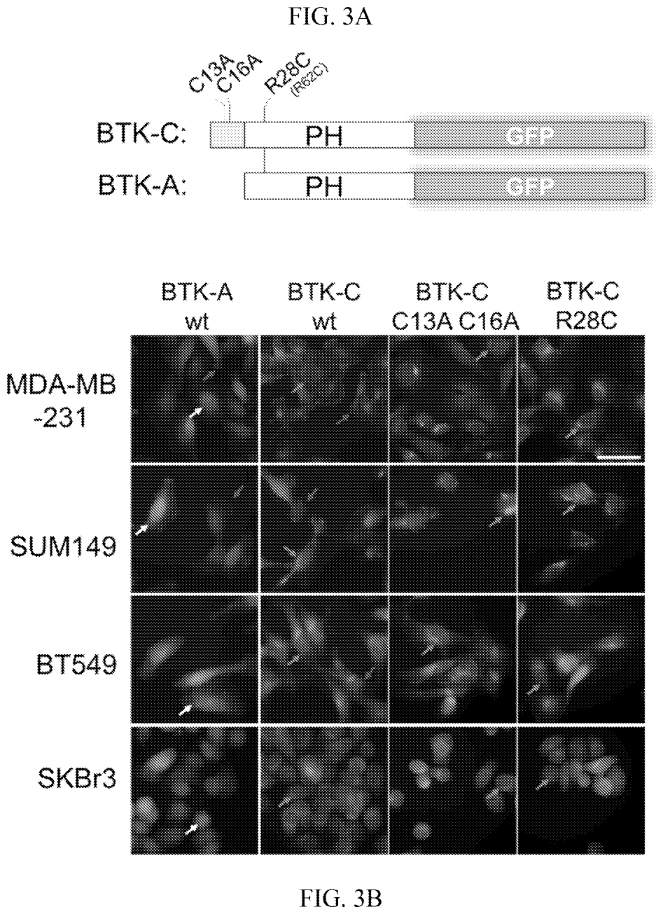

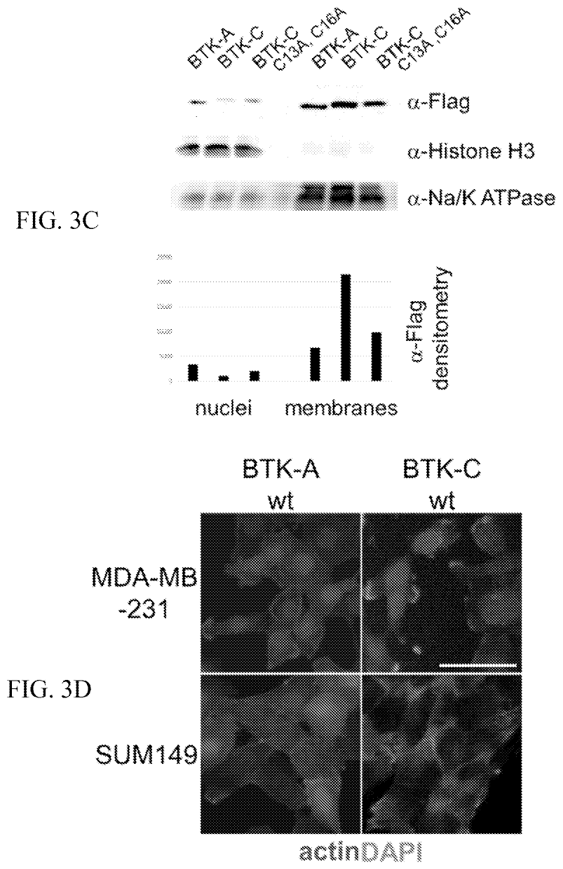

FIG. 3S presents expression of site-directed mutations after transient transfection into HEK 293 cells.

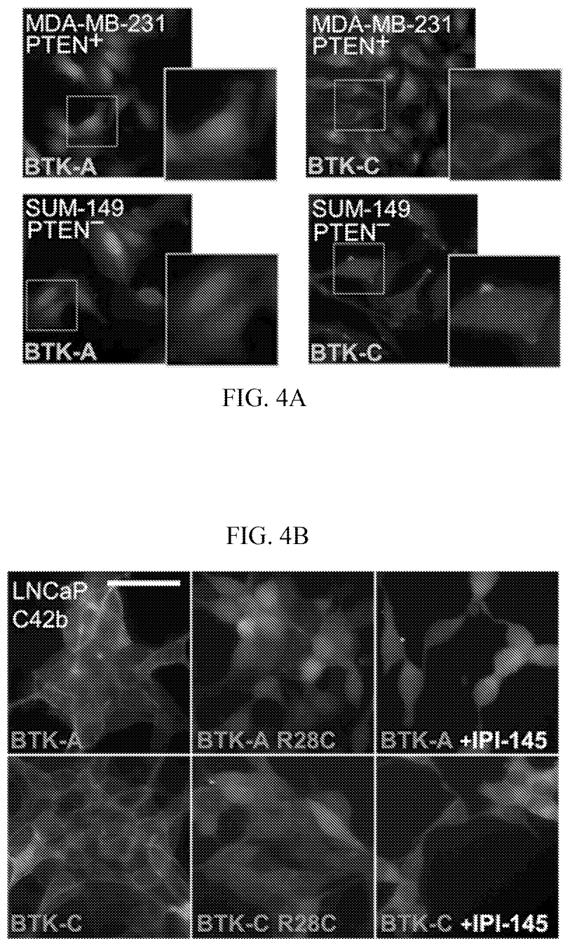

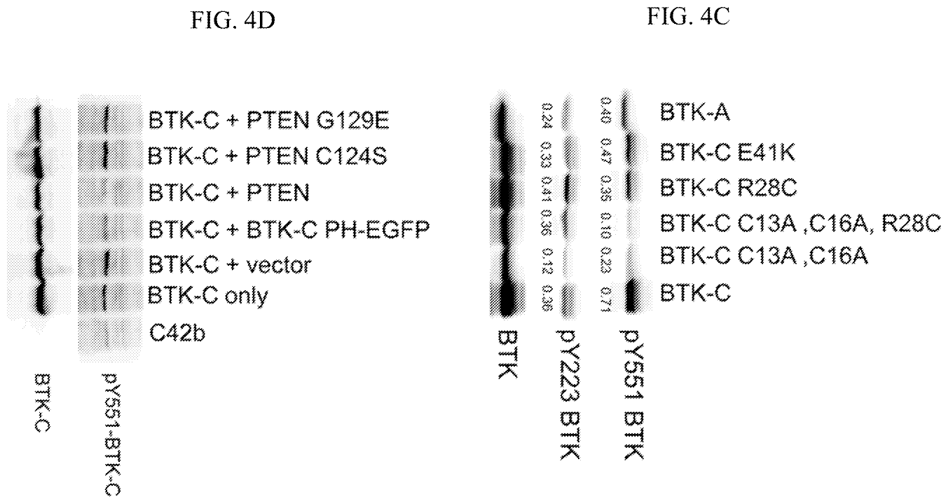

FIG. 4S presents expression of full length BTK constructs in MDA-MB-231 cells. Full immunoblots are shown.

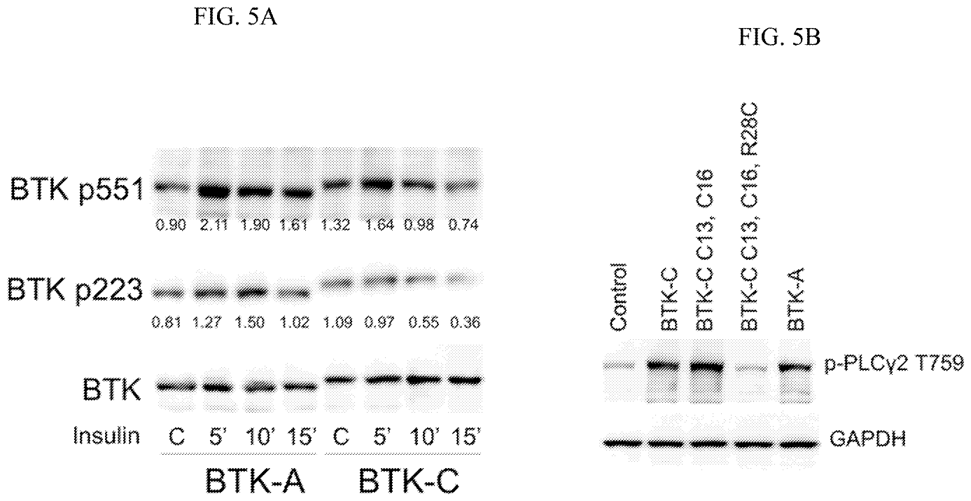

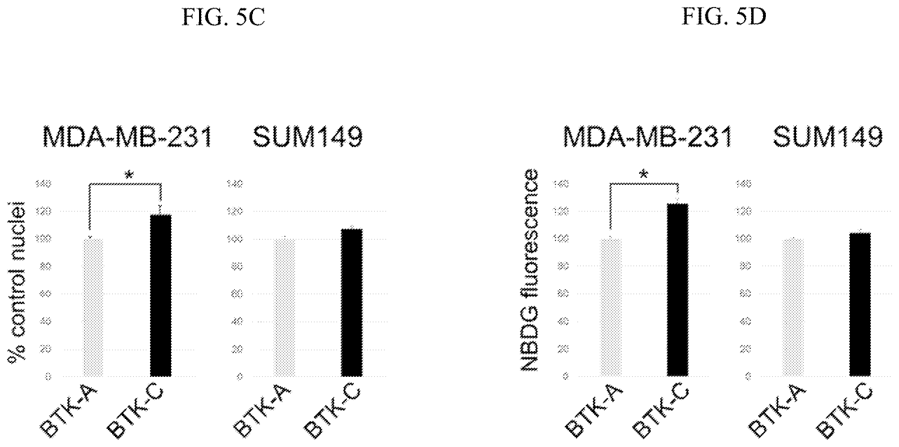

FIG. 5S presents an analysis of all soluble tyrosine kinases and all PH domain-containing serine/threonine kinase genes for the presence of alternative encoded amino termini.

### DEFINITIONS

To facilitate the understanding of this invention a number of terms (set off in quotation marks in this Definitions section) are defined below. Terms defined herein (unless otherwise specified) have meanings as commonly understood by a person of ordinary skill in the areas relevant to the present invention. As used in this specification and its appended claims, terms such as “a”, “an” and “the” are not intended to refer to only a singular entity but include the general class of which a specific example may be used for illustration, unless the context dictates otherwise. The terminology herein is used to describe specific embodiments of the invention, but their usage does not delimit the invention, except as outlined in the claims.

The phrase “chosen from A, B, and C” as used herein, means selecting one or more of A, B, C.

As used herein, absent an express indication to the contrary, the term “or” when used in the expression “A or B,” where A and B refer to a composition, disease, product, etc., means one or the other, or both. As used herein, the term “comprising” when placed before the recitation of steps in a method means that the method encompasses one or more steps that are additional to those expressly recited, and that the additional one or more steps may be performed before, between, and/or after the recited steps. For example, a method comprising steps a, b, and c encompasses a method of steps a, b, x, and c, a method of steps a, b, c, and x, as well as a method of steps x, a, b, and c. Furthermore, the term “comprising” when placed before the recitation of steps in a method does not (although it may) require sequential performance of the listed steps, unless the context clearly dictates otherwise. For example, a method comprising steps a, b, and c encompasses, for example, a method of performing steps in the order of steps a, c, and b, the order of steps c, b, and a, and the order of steps c, a, and b, etc.

Unless otherwise indicated, all numbers expressing quantities of ingredients, properties such as molecular weights, reaction conditions, and so forth as used in the specification and claims are to be understood as being modified in all instances by the term “about.” Accordingly, unless indicated to the contrary, the numerical parameters in the specification and claims are approximations that may vary depending upon the desired properties sought to be obtained by the present invention. At the very least, and without limiting the application of the doctrine of equivalents to the scope of the claims, each numerical parameter should at least be construed in light of the number of reported significant digits and by applying ordinary rounding techniques. Notwithstanding that the numerical ranges and parameters describing the broad scope of the invention are approximations, the numerical values in the specific examples are reported as precisely as possible. Any numerical value, however, inherently contains standard deviations that necessarily result from the errors found in the numerical value's testing measurements.

The term “not” when preceding, and made in reference to, any particularly named molecule (mRNA, etc.) or phenomenon (such as biological activity, biochemical activity, etc.) means that only the particularly named molecule or phenomenon is excluded.

The term “altering” and grammatical equivalents as used herein in reference to the level of any substance and/or phenomenon refers to an increase and/or decrease in the quantity of the substance and/or phenomenon, regardless of whether the quantity is determined objectively, and/or subjectively.

The terms “increase,” “elevate,” “raise,” and grammatical equivalents when used in reference to the level of a substance and/or phenomenon in a first sample relative to a second sample, mean that the quantity of the substance and/or phenomenon in the first sample is higher than in the second sample by any amount that is statistically significant using any art-accepted statistical method of analysis. In one embodiment, the increase may be determined subjectively, for example when a patient refers to their subjective perception of disease symptoms, such as pain, clarity of vision, etc. In another embodiment, the quantity of the substance and/or phenomenon in the first sample is at least 10% greater than the quantity of the same substance and/or phenomenon in a second sample. In another embodiment, the quantity of the substance and/or phenomenon in the first sample is at least 25% greater than the quantity of the same substance and/or phenomenon in a second sample. In yet another embodiment, the quantity of the substance and/or phenomenon in the first sample is at least 50% greater than the quantity of the same substance and/or phenomenon in a second sample. In a further embodiment, the quantity of the substance and/or phenomenon in the first sample is at least 75% greater than the quantity of the same substance and/or phenomenon in a second sample. In yet another embodiment, the quantity of the substance and/or phenomenon in the first sample is at least 90% greater than the quantity of the same substance and/or phenomenon in a second sample. Alternatively, a difference may be expressed as an “n-fold” difference.

The terms “reduce,” “inhibit,” “diminish,” “suppress,” “decrease,” and grammatical equivalents when used in reference to the level of a substance and/or phenomenon in a first sample relative to a second sample, mean that the quantity of substance and/or phenomenon in the first sample is lower than in the second sample by any amount that is statistically significant using any art-accepted statistical method of analysis. In one embodiment, the reduction may be determined subjectively, for example when a patient refers to their subjective perception of disease symptoms, such as pain, clarity of vision, etc. In another embodiment, the quantity of substance and/or phenomenon in the first sample is at least 10% lower than the quantity of the same substance and/or phenomenon in a second sample. In another embodiment, the quantity of the substance and/or phenomenon in the first sample is at least 25% lower than the quantity of the same substance and/or phenomenon in a second sample. In yet another embodiment, the quantity of the substance and/or phenomenon in the first sample is at least 50% lower than the quantity of the same substance and/or phenomenon in a second sample. In a further embodiment, the quantity of the substance and/or phenomenon in the first sample is at least 75% lower than the quantity of the same substance and/or phenomenon in a second sample. In yet another embodiment, the quantity of the substance and/or phenomenon in the first sample is at least 90% lower than the quantity of the same substance and/or phenomenon in a second sample. Alternatively, a difference may be expressed as an “n-fold” difference.

A number of terms herein relate to cancer. “Cancer” is intended herein to encompass all forms of abnormal or improperly regulated reproduction of cells in a subject. “Subject” and “patient” are used herein interchangeably, and a subject may be any mammal but is preferably a human. A “reference subject” herein refers to an individual who does not have cancer. The “reference subject” thereby provides a basis to which another cell (for example a cancer cell) can be compared.

The growth of cancer cells (“growth” herein referring generally to cell division but also to the growth in size of masses of cells) is characteristically uncontrolled or inadequately controlled, as is the death (“apoptosis”) of such cells. Local accumulations of such cells result in a tumor. More broadly, and still denoting “tumors” herein are accumulations ranging from a cluster of lymphocytes at a site of infection to vascularized overgrowths, both benign and malignant. A “malignant” tumor (as opposed to a “benign” tumor) herein comprises cells that tend to migrate to nearby tissues, including cells that may travel through the circulatory system to invade or colonize tissues or organs at considerable remove from their site of origin in the “primary tumor,” so-called herein. Metastatic cells are adapted to penetrate blood vessel wells to enter (“intravasate”) and exit (“extravasate”) blood vessels. Tumors capable of releasing such cells are also referred to herein as “metastatic.” The term is used herein also to denote any cell in such a tumor that is capable of such travel, or that is en route, or that has established a foothold in a target tissue. For example, a metastatic breast cancer cell that has taken root in the lung is referred to herein as a “lung metastasis.” Metastatic cells may be identified herein by their respective sites of origin and destination, such as “breast-to-bone metastatic.” In the target tissue, a colony of metastatic cells can grow into a “secondary tumor,” so called herein.

Primary tumors are thought to derive from a benign or normal cell through a process referred to herein as “cancer progression.” According to this view, the transformation of a normal cell to a cancer cell requires changes (usually many of them) in the cell's biochemistry. The changes are reflected clinically as the disease progresses through stages. Even if a tumor is “clonogenic” (as used herein, an accumulation of the direct descendants of a parent cell), the biochemistry of the accumulating cells changes in successive generations, both because the expression of the genes (controlled by so-called “epigenetic” systems) of these cells becomes unstable and because the genomes themselves change. In normal somatic cells, the genome (that is, all the genes of an individual) is stored in the chromosomes of each cell (setting aside the mitochondrial genome). The number of copies of any particular gene is largely invariant from cell to cell. By contrast, “genomic instability” is characteristic of cancer progression. A genome in a cancer cell can gain (“genomic gain”) or lose (“genomic loss”) genes, typically because an extra copy of an entire chromosome appears (“trisomy”) or a region of a chromosome replicates itself (“genomic gain” or, in some cases, “genomic amplification”) or drops out when the cell divides. Thus, the “copy number” of a gene or a set of genes, largely invariant among normal cells, is likely to change in cancer cells (referred to herein as a “genomic event”), which affects the total expression of the gene or gene set and the biological behavior (“phenotype”) of descendent cells. Thus, in cancer cells, “gene activity” herein is determined not only by the multiple “layers” of epigenetic control systems and signals that call forth expression of the gene but by the number of times that gene appears in the genome. The term “epigenetic” herein refers to any process in an individual that, in operation, affects the expression of a gene or a set of genes in that individual, and stands in contrast to the “genetic” processes that govern the inheritance of genes in successive generations of cells or individuals.

Certain regions of chromosomes, depending upon the specific type of cancer, have proven to be hot spots for genomic gain inasmuch as increases in copy number in the genomes of cells from multiple donors tend to occur in one or a few specific regions of a specific chromosome. Such hot spots are referred to herein as sites of “recurrent genomic gain.” The term is to be distinguished from “recurrent cancer,” which refers to types of cancer that are likely to recur after an initial course of therapy, resulting in a “relapse.” A number of terms herein relate to methods that enable the practitioner to examine many distinct genes at once. By these methods, sets of genes (“gene sets”) have been identified wherein each set has biologically relevant and distinctive properties as a set. Devices (which may be referred to herein as “platforms”) in which each gene in a significant part of an entire genome is isolated and arranged in an array of spots, each spot having its own “address,” enable one to detect, quantitatively, many thousands of the genes in a cell. More precisely, these “microarrays” typically detect expressed genes (an “expressed” gene is one that is actively transmitting its unique biochemical signal to the cell in which the gene resides). Microarray data, inasmuch as they display the expression of many genes at once, permit the practitioner to view “gene expression profiles” in a cell and to compare those profiles cell-to-cell to perform so-called “comparative analyses of expression profiles.” Such microarray-based “expression data” are capable of identifying genes that are “over-expressed” (or under-expressed) in, for example, a disease condition. An over-expressed gene may be referred to herein as having a high “expression score.”

The aforementioned methods for examining gene sets employ a number of well-known methods in molecular biology, to which references are made herein. A gene is a heritable chemical code resident in, for example, a cell, virus, or bacteriophage that an organism reads (decodes, decrypts, transcribes) as a template for ordering the structures of biomolecules that an organism synthesizes to impart regulated function to the organism. Chemically, a gene is a heteropolymer comprised of subunits (“nucleotides”) arranged in a specific sequence. In cells, such heteropolymers are deoxynucleic acids (“DNA”) or ribonucleic acids (“RNA”). DNA forms long strands. Characteristically, these strands occur in pairs. The first member of a pair is not identical in nucleotide sequence to the second strand, but complementary. The tendency of a first strand to bind in this way to a complementary second strand (the two strands are said to “anneal” or “hybridize”), together with the tendency of individual nucleotides to line up against a single strand in a complementarily ordered manner accounts for the replication of DNA.

Experimentally, nucleotide sequences selected for their complementarity can be made to anneal to a strand of DNA containing one or more genes. A single such sequence can be employed to identify the presence of a particular gene by attaching itself to the gene. This so called “probe” sequence is adapted to carry with it a “marker” that the investigator can readily detect as evidence that the probe struck a target. As used herein, the term “marker” relates to any surrogate the artisan may use to “observe” an event or condition that is difficult or impossible to detect directly. In some contexts herein, the marker is said to “target” the condition or event. In other contexts, the condition or event is referred to as the target for the marker. Sequences used as probes may be quite small (e.g., “oligonucleotides” of <20 nucleotides) or quite large (e.g., a sequence of 100,000 nucleotides in DNA from a “bacterial artificial chromosome” or “BAC”). A BAC is a bacterial chromosome (or a portion thereof) with a “foreign” (typically, human) DNA fragment inserted in it. BACs are employed in a technique referred to herein as “fluorescence in situ hybridization” or “FISH.” A BAC or a portion of a BAC is constructed that has (1) a sequence complementary to a region of interest on a chromosome and (2) a marker whose presence is discernible by fluorescence. The chromosomes of a cell or a tissue are isolated (on a glass slide, for example) and treated with the BAC construct. Excess construct is washed away and the chromosomes examined microscopically to find chromosomes or, more particularly, identifiable regions of chromosomes that fluoresce.

Alternatively, such sequences can be delivered in pairs selected to hybridize with two specific sequences that bracket a gene sequence. A complementary strand of DNA then forms between the “primer pair.” In one well-known method, the “polymerase chain reaction” or “PCR,” the formation of complementary strands can be made to occur repeatedly in an exponential amplification. A specific nucleotide sequence so amplified is referred to herein as the “amplicon” of that sequence. “Quantitative PCR” or “qPCR” herein refers to a version of the method that allows the artisan not only to detect the presence of a specific nucleic acid sequence but also to quantify how many copies of the sequence are present in a sample, at least relative to a control. As used herein, “qRTPCR” may refer to “quantitative real-time PCR,” used interchangeably with “qPCR” as a technique for quantifying the amount of a specific DNA sequence in a sample. However, if the context so admits, the same abbreviation may refer to “quantitative reverse transcriptase PCR,” a method for determining the amount of messenger RNA present in a sample. Since the presence of a particular messenger RNA in a cell indicates that a specific gene is currently active (being expressed) in the cell, this quantitative technique finds use, for example, in gauging the level of expression of a gene.

Collectively, the genes of an organism constitute its genome. The term “genomic DNA” may refer herein to the entirety of an organism's DNA or to the entirety of the nucleotides comprising a single gene in an organism. A gene typically contains sequences of nucleotides devoted to coding (“exons”), and non-coding sequences that contribute in one way or another to the decoding process (“introns”).

The term “gene” refers to a nucleic acid (e.g., DNA) comprising covalently linked nucleotide monomers arranged in a particular sequence that comprises a coding sequence necessary for the production of a polypeptide or precursor or RNA (e.g., tRNA, siRNA, rRNA, etc.). The polypeptide can be encoded by a full-length coding sequence or by any portion of the coding sequence so long as the desired activities or functional properties (e.g., enzymatic activity, ligand binding, signal transduction, etc.) of the full-length or fragment are retained. The term also encompasses the coding region together with the sequences located adjacent to the coding region on both the 5′ and 3′ ends, such that the gene corresponds to the length of the full-length mRNA (also referred to as “pre-mRNA,” “nuclear RNA,” or “primary transcript RNA”) transcribed from it. The sequences that are located 5′ of the coding region and are present on the mRNA are referred to as 5′ untranslated sequences. The sequences that are located 3′ or downstream of the coding region and that are present on the mRNA are referred to as 3′ untranslated sequences. The term “gene” encompasses both cDNA (the coding region(s) only) and genomic forms of a gene. A genomic form or clone of a gene contains the coding region, which may be interrupted with non-coding sequences termed “introns” or “intervening regions” or “intervening sequences.” Introns are removed or “spliced out” from the nuclear or primary transcript, and are therefore absent in the messenger RNA (mRNA) transcript. The mRNA functions during translation to specify the sequence or order of amino acids in a nascent polypeptide.

As used herein, the term “palmitoylation” is used to describe the process that modifies specific isoforms of the disclosed proteins. In this regard, palmitoylation is the covalent attachment of fatty acids, e.g., palmitic acid, to cysteine. The precise function of palmitoylation depends on the particular protein being modified. Fatty acids are reversibly bonded to cysteine residues that contain a sulfur atom, which is known as “S-acylation.” Although palmitate can be used in S-acylation of BTK-C, it is possible that other fatty acids or lipids could also be used in this S-acylation. Thus, the embodiments contemplated herein include the possibility that other fatty acids or lipids could be affixed to the products disclosed. Alternatively, as used herein, the term “myristoylation” is used to describe lipid modification involving the addition of myristic acid, to the alpha-amino group of an N-terminal glycine residue. Thus, embodiments disclosed herein contemplate the inclusion of various lipid modifications.

Encoding in DNA (and messenger RNA) is accomplished by 3-membered nucleotide sequences called “codons.” Each codon encrypts an amino acid, and the sequence of codons encrypts the sequence of amino acids that identifies a particular protein. The code for a given gene is embedded in a (usually) much longer nucleotide sequence and is distinguishable to the cell's decoding system from the longer sequence by a “start codon” and a “stop” codon. The decoding system reads the sequence framed by these two codons (the so-called “open reading frame”). The readable code is transcribed into messenger RNA which itself comprises sites that ensure coherent translation of the code from nucleic acid to protein. In particular, the open reading frame is delimited by a so-called “translation initiation” codon and “translation termination” codon.

The term “plasmid” as used herein, refers to a small, independently replicating, piece of DNA. Similarly, the term “naked plasmid” refers to plasmid DNA devoid of extraneous material typically used to effect transfection. As used herein, a “naked plasmid” refers to a plasmid substantially free of calcium-phosphate, DEAE-dextran, liposomes, and/or polyamines. As used herein, the term “purified” refers to molecules (polynucleotides or polypeptides) that are removed from their natural environment, isolated or separated. “Purified” molecules are at least 50% free, preferably at least 75% free, and more preferably at least 90% free from other components with which they are naturally associated.

The term “recombinant DNA” refers to a DNA molecule that is comprised of segments of DNA joined together by means of molecular biology techniques. Similarly, the term “recombinant protein” refers to a protein molecule that is expressed from recombinant DNA.

The term “fusion protein” as used herein refers to a protein formed by expression of a hybrid gene made by combining two gene sequences. Typically this is accomplished by cloning a cDNA into an expression vector in frame (i.e., in an arrangement that the cell can transcribe as a single mRNA molecule) with an existing gene. The fusion partner may act as a reporter (e.g., (Pgal) or may provide a tool for isolation purposes (e.g., GST).

Where an amino acid sequence is recited herein to refer to an amino acid sequence of a protein molecule, “amino acid sequence” and like terms, such as “polypeptide” or “protein” are not meant to limit the amino acid sequence to the complete, native amino acid sequence associated with the recited protein molecule. Rather the terms “amino acid sequence” and “protein” encompass partial sequences, and modified sequences.

The term “wild type” refers to a gene or gene product that has the characteristics of that gene or gene product when isolated from a naturally occurring source. A wild type gene is the variant most frequently observed in a population and is thus arbitrarily designed the “normal” or “wild-type” form of the gene.

In contrast, the terms “modified,” “mutant,” and “variant” (when the context so admits) refer to a gene or gene product that displays modifications in sequence and or functional properties (i.e., altered characteristics) when compared to the wild-type gene or gene product. In some embodiments, the modification comprises at least one nucleotide insertion, deletion, or substitution.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to reduction in binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target. When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “competes for binding” when used in reference to a first and a second polypeptide means that the first polypeptide with an activity binds to the same substrate as does the second polypeptide with an activity. In one embodiment, the second polypeptide is a variant of the first polypeptide (e.g., encoded by a different allele) or a related (e.g., encoded by a homolog) or dissimilar (e.g., encoded by a second gene having no apparent relationship to the first gene) polypeptide. The efficiency (e.g., kinetics or thermodynamics) of binding by the first polypeptide may be the same as or greater than or less than the efficiency of substrate binding by the second polypeptide. For example, the equilibrium binding constant (K.sub.D) for binding to the substrate may be different for the two polypeptides.

As used herein, the term “hybridization” refers to the pairing of complementary nucleic acids. Hybridization and the strength of hybridization (i.e., the strength of the association between the nucleic acids) is impacted by such factors as the degree of complementarity between the nucleic acids, stringency of the conditions involved, the T, of the formed hybrid, and the G: C ratio within the nucleic acids.

As used herein, the term “T,” is used in reference to the “melting temperature.” The melting temperature is the temperature at which a population of double-stranded nucleic acid molecules becomes half dissociated into single strands. The equation for calculating the T.sub.m of nucleic acids is well known in the art. As indicated by standard references, a simple estimate of the T.sub.m value may be calculated by the equation: T.sub.m=81.5+0.41 (% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of T.sub.m.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with 85-100% identity, preferably 70-100% identity). With medium stringency conditions, nucleic acid base pairing will occur between nucleic acids with an intermediate frequency of complementary base sequences (e.g., hybridization under “medium stringency” conditions may occur between homologs with 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution comprising 5×SSPE (43.8 g/l NaCl, 6.9 g/l NaH.sub.2PO.sub.4H.sub.2O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 100 to about 1000 nucleotides in length is employed.

“Medium stringency conditions” when used in reference to nucleic acid hybridization comprise conditions equivalent to binding or hybridization at 42° C. in a solution comprising 5×SSPE (43.8 g/l NaCl, 6.9 g/l NaH.sub.2PO.sub.4H.sub.20 and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 100 to about 1000 nucleotides in length is employed.

“Low stringency conditions” comprise conditions equivalent to binding or hybridization at 42° C. in a solution comprising 5×SSPE (43.8 g/l NaCl, 6.9 g/l NaH.sub.2PO.sub.4H.sub.20 and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5×SSPE, 0.1% SDS at 42° C. when a probe of about 100 to about 1000 nucleotides in length is employed.

The term “equivalent” when made in reference to a hybridization condition as it relates to a hybridization condition of interest means that the hybridization condition and the hybridization condition of interest result in hybridization of nucleic acid sequences which have the same range of percent (%) homology. For example, if a hybridization condition of interest results in hybridization of a first nucleic acid sequence with other nucleic acid sequences that have from 85% to 95% homology to the first nucleic acid sequence, then another hybridization condition is said to be equivalent to the hybridization condition of interest if this other hybridization condition also results in hybridization of the first nucleic acid sequence with the other nucleic acid sequences that have from 85% to 95% homology to the first nucleic acid sequence.

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (Smith and Waterman, Adv. Appl. Math., 2:482, 1981) by the homology alignment algorithm of Needleman and Wunsch (Needleman and Wunsch, I Mol. Biol. 48:443, 1970), by the search for similarity method of Pearson and Lipman (Pearson and Lipman, Proc. Natl. Acad. Sci., U.S.A., 85:2444, 1988), by computerized implementations of these algorithms (GAP, BESTFIT, FASTA, and TFASTA in the Wisconsin Genetics Software Package Release 7.0, Genetics Computer Group, Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, O, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isolenucine; a group of amino acids having aliphatic hydroxyl side chains is serine and threonine; a group of amino acids having amide-containing side chains is asparagine and glutamine; a group of amino acids having aromatic side chains is phenylalanine, tyrosine, and tryptophan; a group of amino acids having acidic side chains is glutamic acid and aspartic acid; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

“Amplification” is used herein in two different ways. A given gene typically appears in a genome once, on one chromosome. Since chromosomes in somatic cells of eukaryotes are in general paired, two copies or alleles of each gene are found. In some conditions, such as cancer, replication of chromosome pairs during cell division is disturbed so that multiple copies of a gene or chromosome accrue over successive generations. The phenomenon is referred to generally (and herein) as “amplification.”

In the context of molecular biological experimentation, the term is used differently. Experimentally, “amplification” is used in relation to a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under the conditions in which they are used, will process only specific sequences of nucleic acids in a heterogeneous mixture of nucleic acids. In particular, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences.

As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

As used herein, the term “polymerase chain reaction” (“PCR”) refers to the method of Mullis (U.S. Pat. Nos. 4,683,195, 4,683,202, and 4,965,188, hereby incorporated by reference), that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

As used herein, the terms “restriction endonucleases” and “restriction enzymes” refer to bacterial enzymes, each of which cut double-stranded DNA at or near a specific nucleotide sequence.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding gene includes, by way of example, such nucleic acid in cells ordinarily expressing gene where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

The terms “fragment” and “portion” when used in reference to a nucleotide sequence (as in “a portion of a given nucleotide sequence”) refers to partial segments of that sequence. The fragments may range in size from four nucleotides to the entire nucleotide sequence minus one nucleotide (10 nucleotides, 20, 30, 40, 50, 100, 200, etc.).

Similarly, the terms “fragment” and “portion” when used in reference to a polypeptide sequence refers to partial segments of that sequence. In some embodiments, the portion has an amino-terminal and/or carboxy-terminal deletion as compared to the native protein, but where the remaining amino acid sequence is identical to the corresponding positions in the amino acid sequence deduced from a full-length cDNA sequence. Fragments are preferably at least 4 amino acids long, more preferably at least 50 amino acids long, and most preferably at least 50 amino acids long or longer (the entire amino acid sequence minus on amino acid). In particularly preferred embodiments, the portion comprises the amino acid residues required for intermolecular binding of the compositions of the present invention with its various ligands and/or substrates.

As used herein the term “portion” when in reference to a protein (as in “a portion of a given protein”) refers to fragments of that protein. The fragments may range in size from four consecutive amino acid residues to the entire amino acid sequence minus one amino acid.

As used herein the term “coding region” when used in reference to structural gene refers to the nucleotide sequences that encode the amino acids found in the nascent polypeptide as a result of translation of a mRNA molecule. The coding region is bounded, in eukaryotes, on the 5′ side by the nucleotide triplet “ATG” that encodes the initiator methionine and on the 3′ side by one of the three triplets which specify stop codons (i.e., TAA, TAG, TGA).

The term “recombinant DNA molecule” as used herein refers to a DNA molecule that is comprised of segments of DNA joined together by means of molecular biological techniques.

Similarly, the term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences, that are the native protein contains only those amino acids found in the protein as it occurs in nature. A native protein may be produced by recombinant means or may be isolated from a naturally occurring source.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (Sambrook et al., Molecular Cloning: A Laboratory Manual, Cold Spring Harbor Press, NY, pp. 9.31-9.58, 1989).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (Sambrook, et al., supra, pp. 7.39-7.52, 1989).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabelled antibodies

As used herein, the term “transgenic” refers to a cell or organism whose genome has been heritably altered by genetically engineering into the genome a gene (“transgene”) not normally part of it or removing from it a gene ordinarily present (a “knockout” gene). The “transgene” or “foreign gene” may be placed into an organism by introducing it into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term host cell refers to any eukaryotic or prokaryotic cell (e.g. bacterial cells such as E. coli, yeast cells, mammalian cells, avian cells, amphibian cells, plant cells, fish cells, and insect cells), whether located in vitro or in vivo. For example, host cells may be located in a transgenic animal.

The term “transfection” as used herein refers to the introduction of foreign DNA into eukaryotic cells. Transfection may be accomplished by a variety of means known to the art including calcium phosphate-DNA co-precipitation, DEAE-dextran-transfection, electroporation, mediated transfection, polybrene-mediated microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell in the sense that the foreign DNA will be passed on to daughter cells. The term encompasses transfections of foreign DNA into the cytoplasm only. In general, however, the foreign DNA reaches the nucleus of the transfected cell and persists there for several days. During this time the foreign DNA is subject to the regulatory controls that govern the expression of endogenous genes in the chromosomes. The term “transient transfectant” refers to cells that have taken up foreign DNA but have failed to integrate this DNA. The term “transient transfection” encompasses transfection of foreign DNA into the cytoplasm only

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of is modified to optimize conditions for particular types of cells. The art is well aware of these numerous modifications.

A “composition comprising a given polynucleotide sequence” as used herein refers broadly to any composition containing the given polynucleotide sequence.

Such compositions may be employed as hybridization probes, typically in an aqueous solution containing salts (e.g., NaCl), detergents (e.g., SDS), and other components (e.g., Denhardt's solution, dry milk, salmon sperm DNA, etc.).

The terms “N-terminus” “NH₂-terminus” and “amino-terminus” refer to the amino acid residue corresponding to the methionine encoded by the start codon (e.g., position or residue 1). In contrast the terms “C-terminus” “COOH-terminus” and “carboxy terminus” refer to the amino acid residue encoded by the final codon (e.g., last or final residue prior to the stop codon).

The term “conservative substitution” as used herein refers to a change that takes place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur-containing (cysteine and methionine). Whether a change in the amino acid sequence of a peptide results in a functional homolog can be readily determined by assessing the ability of the variant peptide to function in a fashion similar to the wild-type protein. Peptides having more than one replacement can readily be tested in the same manner. In contrast, the term “non-conservative substitution” refers to a change in which an amino acid from one family is replaced with an amino acid from another family (e.g., replacement of a glycine with a tryptophan). Guidance in determining which amino acid residues can be substituted, inserted, or deleted without abolishing biological activity can be found using computer programs (e.g., LASERGENE software, DNASTAR Inc., Madison, Wis.

A peptide sequence and nucleotide sequence may be “endogenous” or “heterologous” (i.e., “foreign”). The term “endogenous” refers to a sequence which is naturally found in the cell or virus into which it is introduced so long as it does not contain some modification relative to the naturally-occurring sequence. The term “heterologous” refers to a sequence which is not endogenous to the cell or virus into which it is introduced. For example, heterologous DNA includes a nucleotide sequence which is ligated to, or is manipulated to become ligated to, a nucleic acid sequence to which it is not ligated in nature, or to which it is ligated at a different location in nature. Heterologous DNA also includes a nucleotide sequence which is naturally found in the cell or virus into which it is introduced and which contains some modification relative to the naturally-occurring sequence. Generally, although not necessarily, heterologous DNA encodes heterologous RNA and heterologous proteins that are not normally produced by the cell or virus into which it is introduced. Examples of heterologous DNA include reporter genes, transcriptional and translational regulatory sequences, DNA sequences which encode selectable marker proteins (e.g., proteins which confer drug resistance), etc. In preferred embodiments, the terms “heterologous antigen” and “heterologous sequence” refer to a non-hepadna virus antigen or amino acid sequence including but not limited to microbial antigens, mammalian antigens and allergen antigens.

The terms “peptide,” “peptide sequence,” “amino acid sequence,” “polypeptide,” and “polypeptide sequence” are used interchangeably herein to refer to at least two amino acids or amino acid analogs which are covalently linked by a peptide bond or an analog of a peptide bond. The term peptide includes oligomers and polymers of amino acids or amino acid analogs. The term peptide also includes molecules which are commonly referred to as peptides, which generally contain from about two (2) to about twenty (20) amino acids. The term peptide also includes molecules which are commonly referred to as polypeptides, which generally contain from about twenty (20) to about fifty amino acids (50). The term peptide also includes molecules which are commonly referred to as proteins, which generally contain from about fifty (50) to about three thousand (3000) amino acids. The amino acids of the peptide may be L-amino acids or D-amino acids. A peptide, polypeptide or protein may be synthetic, recombinant or naturally occurring. A synthetic peptide is a peptide which is produced by artificial means in vitro

The terms “oligosaccharide” and “OS” antigen refer to a carbohydrate comprising up to ten component sugars, either 0 or N linked to the next sugar. Likewise, the terms “polysaccharide” and “PS” antigen refer to polymers of more than ten monosaccharide residues linked glycosidically in branched or unbranched chains

As used herein, the term “mammalian sequence” refers to synthetic, recombinant or purified sequences (preferably sequence fragments comprising at least one B cell epitope) of a mammal. Exemplary mammalian sequences include cytokine sequence, MHC class I heavy chain sequences, MHC class II alpha and beta chain sequences, and amyloid 13-peptide sequences.

The terms “mammals” and “mammalian” refer animals of the class mammalia which nourish their young by fluid secreted from mammary glands of the mother, including human beings. The class “mammalian” includes placental animals, marsupial animals, and monotrematal animals. An exemplary “mammal” may be a rodent, primate (including simian and human) ovine, bovine, ruminant, lagomorph, porcine, caprine, equine, canine, feline, ave, etc. Preferred non-human animals are selected from the order Rodentia.

Preferred embodiments of the present invention are primarily directed to vertebrate (backbone or notochord) members of the animal kingdom.

The terms “patient” and “subject” refer to a mammal that may be treated using the methods of the present invention.

The term “control” refers to subjects or samples which provide a basis for comparison for experimental subjects or samples. For instance, the use of control subjects or samples permits determinations to be made regarding the efficacy of experimental procedures. In some embodiments, the term “control subject” refers to a subject that which receives a mock treatment (e.g., saline alone).

The terms “diluent” and “diluting agent” as used herein refer to agents used to diminish the strength of an admixture. Exemplary diluents include water, physiological saline solution, human serum albumin, oils, polyethylene glycols, glycerine, propylene glycol or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

The terms “carrier” and “vehicle” as used herein refer to usually inactive accessory substances into which a pharmaceutical substance is suspended. Exemplary carriers include liquid carriers (such as water, saline, culture medium, saline, aqueous dextrose, and glycols) and solid carriers (such as carbohydrates exemplified by starch, glucose, lactose, sucrose, and dextrans, anti-oxidants exemplified by ascorbic acid and glutathione, and hydrolyzed proteins.

The term “pharmaceutically acceptable salts” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

As used herein, the term “pharmaceutical composition” is intended to include the combination of an active agent with a carrier, inert or active, making the composition suitable for diagnostic or therapeutic use in vitro, in vivo, or ex vivo.

The term “derived” when in reference to a peptide derived from a source (such as a microbe, cell, etc.) as used herein is intended to refer to a peptide which has been obtained (e.g., isolated, purified, etc.) from the source. Alternatively, or in addition, the peptide may be genetically engineered and/or chemically synthesized.

The terms “operably linked,” “in operable combination” and “in operable order” as used herein refer to the linkage of nucleic acid sequences such that they perform their intended function. For example, operably linking a promoter sequence to a nucleotide sequence of interest refers to linking the promoter sequence and the nucleotide sequence of interest in a manner such that the promoter sequence is capable of directing the transcription of the nucleotide sequence of interest and/or the synthesis of a polypeptide encoded by the nucleotide sequence of interest.

Similarly, operably linking a nucleic acid sequence encoding a protein of interest means linking the nucleic acid sequence to regulatory and other sequences in a manner such that the protein of interest is expressed. The term also refers to the linkage of amino acid sequences in such a manner so that a functional protein is produced.

The terms “C-terminal portion,” “COOH-terminal portion,” “carboxy terminal portion,” “C-terminal domain,” “COOH-terminal domain,” and “carboxy terminal domain,” when used in reference to an amino acid sequence of interest refer to the amino acid sequence (and portions thereof that is located from approximately the middle of the amino acid sequence of interest to the C-terminal-most amino acid residue of the sequence of interest. The terms “specific binding,” “binding specificity,” and grammatical equivalents thereof when made in reference to the binding of a first molecule (such as a polypeptide, glycoprotein, nucleic acid sequence, etc.) to a second molecule (such as a polypeptide, glycoprotein, nucleic acid sequence, etc.) refer to the preferential interaction between the first molecule with the second molecule as compared to the interaction between the second molecule with a third molecule. Specific binding is a relative term that does not require absolute specificity of binding; in other words, the term “specific binding” does not require that the second molecule interact with the first molecule in the absence of an interaction between the second molecule and the third molecule. Rather, it is sufficient that the level of interaction between the first molecule and the second molecule is higher than the level of interaction between the second molecule with the third molecule. “Specific binding” of a first molecule with a second molecule also means that the interaction between the first molecule and the second molecule is dependent upon the presence of a particular structure on or within the first molecule; in other words the second molecule is recognizing and binding to a specific structure on or within the first molecule rather than to nucleic acids or to molecules in general. For example, if a second molecule is specific for structure “A” that is on or within a first molecule, the presence of a third nucleic acid sequence containing structure A will reduce the amount of the second molecule which is bound to the first molecule.

For example, the term “has the biological activity of a specifically named protein” when made in reference to the biological activity of a variant of the specifically named protein refers, for example, to a quantity of binding of an antibody that is specific for the specifically named protein to the variant which is preferably greater than 50% (preferably from 50% to 500%, more preferably from 50% to 200%, most preferably from 50% to 100%), as compared to the quantity of binding of the same antibody to the specifically named protein.

Reference herein to any specifically named nucleotide sequence includes within its scope fragments, homologs, and sequences that hybridize under stringent condition to the specifically named nucleotide sequence. The term “homolog” of a specifically named nucleotide sequence refers to an oligonucleotide sequence which exhibits greater than or equal to 50% identity to the sequence of interest. Alternatively, or in addition, a homolog of any specifically named nucleotide sequence is defined as an oligonucleotide sequence which has at least 95% identity with the sequence of the nucleotide sequence in issue. In another embodiment, the sequence of the homolog has at least 90% identity, and preferably at least 85% identity with the sequence of the nucleotide sequence in issue.

Exons, introns, genes and entire gene-sets are characteristically locatable with respect to one another. That is, they have generally invariant “genomic loci” or “genomic positions.” Genes distributed across one or several chromosomes can be mapped to specific locations on specific chromosomes. The field of “cytogenetics” addresses several aspects of gene mapping. First, optical microscopy reveals features of chromosomes that are useful as addresses for genes. In humans, chromosomes are morphologically distinguishable from one another and each (except for the Y-chromosome) has two distinct arms separated by a “centromere.” Each arm has distinctive “bands” occupied by specific genes. Disease-related changes in chromosome number and changes in banding form the basis for diagnosing a number of diseases. “Microdissection” of chromosomes and DNA analysis of the microdissected fragments have connected specific DNA sequences to specific locations on chromosomes. In cancer, a region of a chromosome may duplicate or amplify itself or drop out entirely. FISH, mentioned above, and “comparative genomic hybridization” (“CGH”) have extended the reach of cytogenetic analysis to the extent of measuring genome alterations within and between individuals. CGH, for example, in which chromosomes from a normal cell are hybridized with a corresponding preparation from a cancer cell provides a means of directly determining cancer-related differences in copy number of chromosomal regions.

“Targeted therapeutics” is used herein to denote any therapeutic modality that affects only or primarily only the cells or tissues selected (“targeted”) for treatment. A monoclonal antibody specific for an antigen expressed only by a target (if retained by the target) is highly useful in targeted therapeutics. In the case of unwanted cells such as cancer cells, if the antibody doesn't induce destruction of the target directly, it may do so indirectly by carrying to the target, for example, an agent coupled to the antibody. On the other hand, agents that suppress processes that tend to promote uncontrolled proliferation of cells (“antineoplastic agents”) can be delivered to target sites in this manner.

The term “agent” is used herein in its broadest sense to refer to a composition of matter, a process or procedure, a device or apparatus employed to exert a particular effect. By way of non-limiting example, a surgical instrument may be employed by a practitioner as an “excising” agent to remove tissue from a subject; a chemical may be used as a pharmaceutical agent to remove, damage or neutralize the function of a tissue, etc. Such pharmaceutical agents are said to be “anticellular.” Cells may be removed by an agent that promotes apoptosis. A variety of toxic agents, including other cells (e.g., cytotoxic T-cell lymphocytes) and their secretions, and a plethora of chemical species, can damage cells.

The term “by-stander”, as used herein, refers to a process or event initiated or affected by another, causative event or process

The term “knockdown”, as used herein, refers to a method of selectively preventing the expression of a gene in an individual.

The term “oncogene”, as used herein, refers to any gene that regulates a process affecting the suppression of abnormal proliferative events.

The term “single nucleotide polymorphism” or “SNP”, as used herein, refers to a DNA sequence variation occurring when a single nucleotide in the genome (or other shared sequence) differs between members of a species or between paired chromosomes in an individual. Single nucleotide polymorphisms may fall within coding sequences of genes, non-coding regions of genes, or in the intergenic regions between genes. Single nucleotide polymorphisms within a coding sequence will not necessarily change the amino acid sequence of the protein that is produced, due to degeneracy of the genetic code. A Single nucleotide polymorphism in which both forms lead to the same polypeptide sequence is termed synonymous (sometimes called a silent mutation)—if a different polypeptide sequence is produced they are non-synonymous. Single nucleotide polymorphisms that are not in protein-coding regions may still have consequences for gene splicing, transcription factor binding, or the sequence of non-coding RNA.

The term “tissue array” or “tissue microarray”, as used herein, refers to high throughput platforms for the rapid analysis of protein, RNA, or DNA molecules. These arrays can be used to validate the clinical relevance of potential biological targets in the development of diagnostics, therapeutics and to study new disease markers and genes. Tissue arrays are suitable for genomics-based diagnostic and drug target discovery.

As used herein, the term “shRNA” or “short hairpin RNA” refers to a sequence of ribonucleotides comprising a single-stranded RNA polymer that makes a tight hairpin turn on itself to provide a “double-stranded” or duplexed region. shRNA can be used to silence gene expression via RNA interference. shRNA hairpin is cleaved into short interfering RNAs (siRNA) by the cellular machinery and then bound to the RNA-induced silencing complex (RISC). It is believed that the complex inhibits RNA as a consequence of the complexed siRNA hybridizing to and cleaving RNAs that match the siRNA that is bound thereto.

As used herein, the term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi inhibits the gene by compromising the function of a target RNA, completely or partially. Both plants and animals mediate RNAi by the RNA-induced silencing complex (RISC); a sequence-specific, multicomponent nuclease that destroys messenger RNAs homologous to the silencing trigger. RISC is known to contain short RNAs (approximately 22 nucleotides) derived from the double-stranded RNA trigger, although the protein components of this activity are unknown. However, the 22-nucleotide RNA sequences are homologous to the target gene that is being suppressed. Thus, the 22-nucleotide sequences appear to serve as guide sequences to instruct a multicomponent nuclease, RISC, to destroy the specific mRNAs. Carthew has reported (Curr. Opin. Cell Biol. 13 (2): 244-248 (2001)) that eukaryotes silence gene expression in the presence of dsRNA homologous to the silenced gene. Biochemical reactions that recapitulate this phenomenon generate RNA fragments of 21 to 23 nucleotides from the double-stranded RNA. These stably associate with an RNA endonuclease, and probably serve as a discriminator to select mRNAs. Once selected, mRNAs are cleaved at sites 21 to 23 nucleotides apart.

As used herein, the term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand”; the strand homologous to the target RNA molecule is the “sense strand” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “xenograft”, as used herein, refers to the transfer or transplant of a cell(s) or tissue from one species to an unlike species (or genus or family).

The term “orthotopic” or “orthotopic xenograft”, as used herein, refers to a cell or tissue transplant grafted into its normal place in the body.

The term “fluorescent activated cell sorting” or “FACS”, as used herein, refers to a technique for counting, examining, and sorting microscopic particles suspended in a stream of fluid. It allows simultaneous multiparametric analysis of the physical and/or chemical characteristics of single cells flowing through an optical and/or electronic detection apparatus. Generally, a beam of light (usually laser light) of a single wavelength is directed onto a hydro dynamically focused stream of fluid. A number of detectors are aimed at the point where the stream passes through the light beam; one in line with the light beam (Forward Scatter, correlates to cell volume) and several perpendicular to the beam, (Side Scatter, correlates to the inner complexity of the particle and/or surface roughness) and one or more fluorescent detectors. Each suspended particle passing through the beam scatters the light in some way, and fluorescent chemicals found in the particle or attached to the particle may be excited into emitting light at a lower frequency than the light source. By analyzing the combinations of scattered and fluorescent light picked up by the detectors it is then possible to derive information about the physical and chemical structure of each individual particle.

The term “data mining”, as used herein, refers to the automated or convenient extraction of patterns representing knowledge implicitly stored or captured in large databases, data warehouses, internet websites, other massive information repositories, or data streams.

The terms “over-express”, “over-expressing” and grammatical equivalents, as used herein, refer to the production of a gene product at levels that exceed production in normal or control cells. The term “over-expression” or “highly expressed” may be specifically used in reference to levels of mRNA to indicate a higher level of expression than that typically observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed, the amount of 28S rRNA (an abundant RNA transcript present at essentially the same amount in all tissues) present in each sample can be used as a means of normalizing or standardizing the mRNA-specific signal observed on Northern blots. Over-expression may likewise result in elevated levels of proteins encoded by said mRNAs.

The term “heatmap”, as used herein, refers to a graphical representation of data where the values obtained from a variable two-dimensional map are represented as colors. As related to the field of molecular biology, heat maps typically represent the level of expression of multiple genes across a number of comparable samples as obtained from a microarray.

The term “phage display”, as used herein, refers to the integration/ligation of numerous genetic sequences from a DNA library, consisting of all coding sequences of a cell, tissue or organism library into the genome of a bacteriophage (i.e. phage) for high-throughput screening protein-protein and/or protein-DNA interactions. Using a multiple cloning site, these fragments are inserted in all three possible reading frames to ensure that the cDNA is translated. DNA fragments are then expressed on the surface of the phage particle as part of it coat protein. The phage gene and insert DNA hybrid is then amplified by transforming bacterial cells (such as TGI E. coli cells), to produce progeny phages that display the relevant protein fragment as part of their outer coat. By immobilizing relevant DNA or protein target(s) to the surface of a well, a phage that displays a protein that binds to one of those targets on its surface will remain while others are removed by washing. Those that remain can be eluted, used to produce more phage (by bacterial infection with helper phage) and so produce an enriched phage mixture. Phage eluted in the final step can be used to infect a suitable bacterial host, from which the phagemids can be collected and the relevant DNA sequence excised and sequenced to identify the relevant, interacting proteins or protein fragments.

The term “apoptosis”, as used herein, refers to a form of programmed cell death in multicellular organisms that involves a series of biochemical events that lead to a variety of morphological changes, including blebbing, changes to the cell membrane such as loss of membrane asymmetry and attachment, cell shrinkage, nuclear fragmentation, chromatin condensation, and chromosomal DNA fragmentation. Defective apoptotic processes have been implicated in an extensive variety of diseases; for example, defects in the apoptotic pathway have been implicated in diseases associated with uncontrolled cell proliferations, such as cancer.

The term “bioluminescence imaging” or “BLI”, as used herein, refers to the noninvasive study of ongoing biological processes in living organisms (for example laboratory animals) using bioluminescence, the process of light emission in living organisms. Bioluminescence imaging utilizes native light emission from one of several organisms which bioluminescence. The three main sources are the North American firefly, the sea pansy (and related marine organisms), and bacteria like Photorhabdus luminescens and Vibrio fischeri. The DNA encoding the luminescent protein is incorporated into the laboratory animal either via a virus or by creating a transgenic animal. While the total amount of light emitted via bioluminescence is typically small and not detected by the human eye, an ultra-sensitive CCD camera can image bioluminescence from an external vantage point. Common applications of BLI include in vivo studies of infection (with bioluminescent pathogens), cancer progression (using a bioluminescent cancer cell line), and reconstitution kinetics (using bioluminescent stem cells).

The term “consensus region” or “consensus sequence”, as used herein, refers to the conserved sequence motifs that show which nucleotide residues are conserved and which nucleotide residues are variable when comparing multiple DNA, RNA, or amino acid sequence alignments. When comparing the results of a multiple sequence alignment, where related sequences are compared to each other, and similar functional sequence motifs are found. The consensus sequence shows which residues are conserved (are always the same), and which residues are variable. A consensus sequence may be a short sequence of nucleotides, which is found several times in the genome and is thought to play the same role in its different locations. For example, many transcription factors recognize particular consensus sequences in the promoters of the genes they regulate. In the same way restriction enzymes usually have palindromic consensus sequences, usually corresponding to the site where they cut the DNA. Splice sites (sequences immediately surrounding the exon-intron boundaries) can also be considered as consensus sequences. In one aspect, a consensus sequence defines a putative DNA recognition site, obtained for example, by aligning all known examples of a certain recognition site and defined as the idealized sequence that represents the predominant base at each position. Related sites should not differ from the consensus sequence by more than a few substitutions.

The term “linkage”, or “genetic linkage,” as used herein, refers to the phenomenon that particular genetic loci of genes are inherited jointly. The “linkage strength” refers to the probability of two genetic loci being inherited jointly. As the distance between genetic loci increases, the loci are more likely to be separated during inheritance, and thus linkage strength is weaker.

The term “neighborhood score”, as used herein, refers to the relative value assigned to a genomic locus based on a geometry-weighted sum of expression scores of all the genes on a given chromosome, as a measurement of the copy number status of the locus. A positive neighborhood score is indicative of an increase in copy number, whereas a negative neighborhood score is indicative of a decrease in copy number.

The term “expression score”, as used herein, refers to the expression differences (i.e., the level of transcription (RNA) or translation (protein)) between comparison groups on a given chromosome. The expression score for a given gene is calculated by correlating the level of expression of said gene with a phenotype in comparison. For example, an expression score may represent a comparison of the expression differences of a given gene in normal vs. abnormal conditions, such as parental vs. drug-resistant cell lines. As used herein, the term “regional expression score” refers to the expression score of gene(s) in proximity to the locus in consideration. Since linkage strength between genetic loci decreases (i.e. decays) as the distance between them increases, the “regional expression score” more accurately reflects the expression differences between comparison groups by assigning greater weight to the expression scores of genes in proximity to the locus in consideration.

The terms “geometry-weighted” or “geometry-weighted sum”, as used herein, refers to the significance attached to a given value, for example an “expression score”, based on physical position, including but not limited to genomic position. Since linkage strength between genetic loci decreases (i.e. decays) as the distance between them increases, the “weight” assigned to a given value is adjusted accordingly.

The term “copy number alteration” or “CNA”, as used herein, refers to the increase (i.e. genomic gain) or decrease (i.e. genomic loss) in the number of copies of a gene at a specific locus of a chromosome as compared to the “normal” or “standard” number of copies of said gene that locus. As used herein, an increase in the number of copies of a given gene at a specific locus may also be referred to as an “amplification” or “genomic amplification” and should not be confused with the use of the term “amplification” as it relates, for example, to amplification of DNA or RNA in PCR and other experimental techniques.

The term “clonogenic assay”, as used herein, refers to a technique for studying whether a given cancer therapy (for example drugs or radiation) can reduce the clonogenic survival and proliferation of tumor cells. While any type of cell may be used, human tumor cells are commonly used for oncological research. The term “clonogenic” refers to the fact that these cells are clones of one another.

The term “adjuvant therapy”, as used herein, refers to additional treatment given after the primary treatment to increase the chances of a cure. In some instances, adjuvant therapy is administered after surgery where all detectable disease has been removed, but where there remains a statistical risk of relapse due to occult disease. If known disease is left behind following surgery, then further treatment is not technically “adjuvant”. Adjuvant therapy may include chemotherapy, radiation therapy, hormone therapy, or biological therapy. For example, radiotherapy or chemotherapy is commonly given as adjuvant treatment after surgery for a breast cancer. Oncologists use statistical evidence to assess the risk of disease relapse before deciding on the specific adjuvant therapy. The aim of adjuvant treatment is to improve disease-specific and overall survival. Because the treatment is essentially for a risk, rather than for provable disease, it is accepted that a proportion of patients who receive adjuvant therapy will already have been cured by their primary surgery. Adjuvant chemotherapy and radiotherapy are often given following surgery for many types of cancer, including colon cancer, lung cancer, pancreatic cancer, breast cancer, prostate cancer, and some gynecological cancers.

The term “matched samples”, as used herein, as for example “matched cancer samples” refers to a sample in which individual members of the sample are matched with every other sample by reference to a particular variable or quality other than the variable or quality immediately under investigation. Comparison of dissimilar groups based on specified characteristics is intended to reduce bias and the possible effects of other variables. Matching may be on an individual (matched pairs) or a group-wide basis.

The term “genomic segments”, as used herein, refers to any defined part or region of a chromosome, and may contain zero, one or more genes.

The term “co-administer”, as used herein, refers to the administration of two or more agents, drugs, and/or compounds together (i.e. at the same time).

The term “diagnose” or “diagnosis”, as used herein, refers to the determination, recognition, or identification of the nature, cause, or manifestation of a condition based on signs, symptoms, and/or laboratory findings.

The term “resistance”, as used herein, refers to cancer cells that do not respond to chemotherapy drugs (i.e. chemotherapeutic agents). Typically, a first course of chemotherapy may prove highly beneficial, nearly annihilating a tumor, but a few resistant cancer cells often survive and proliferate. Too often, despite more aggressive second and third courses of chemotherapy, the remaining drug-defiant cells thrive, displaying increasing resistance to drug therapy and eventually displaying virtual invulnerability to chemotherapy. After the drug's effectiveness fades, the patient relapses. This occurs in patients with a variety of blood cancers and solid tumors, including breast, ovarian, lung, and lower gastrointestinal tract cancers. Nature Biotechnology 18: IT18-IT20 (2000). Resistance to treatment with anticancer drugs results from a variety of factors including individual variations in patients and somatic cell genetic differences in tumors, even those from the same tissue of origin. Frequently resistance is intrinsic to the cancer, but as therapy becomes more and more effective, acquired resistance has also become common. The development of multidrug resistance (MDR) to chemotherapy remains a major challenge in the treatment of cancer. Resistance exists against every effective anticancer drug and can develop by numerous mechanisms including decreased drug uptake, increased drug efflux, activation of detoxifying systems, activation of DNA repair mechanisms, and insensitivity to drug-induced apoptosis. Methods Mol. Biol. 596:47-76 (2010).

In some embodiments, the present invention contemplates treating drug resistant cancer cells. It is not intended that the present invention be limited to the degree of resistance, i.e. resistance can be shown simply by the fact that it takes higher doses of drug to kill these cells. The cells need not be resistant at every dose. The cells may be resistant such that higher doses needed to kill the cells will not be well tolerated by the patient.

As used herein, “Doxorubicin” (trade name Doxil) also known as “hydroxydaunorubicin” or “Adriamycin” refers to a drug used in cancer chemotherapy, that is considered to be the most effective agent in the treatment of breast cancer patients. Doxorubicin is an anthracycline antibiotic, closely related to the natural product daunomycin, and like all anthracyclines, works by intercalating DNA, with the most serious adverse effect being life-threatening heart damage. Doxorubicin is commonly used in the treatment of a wide range of cancers, including some leukemia's and Hodgkin's lymphoma, as well as cancers of the bladder, breast, stomach, lung, ovaries, thyroid, soft tissue sarcoma, multiple myeloma. It is frequently used in breast cancer therapy either as single-agent or in combination with other drugs like docetaxel and cyclophosphamide. Unfortunately, resistance to this agent is common, representing a major obstacle to successful treatment. Mol. Cancer Ther. 5(8):2115-20 (2006). Doxorubicin is administered intravenously, as the hydrochloride salt. It may be sold under the brand names Adriamycin PFS, Adriamycin RDF, or Rubex. Commonly used doxorubicin-containing regimens include, but are not necessarily limited to, AC (Adriamycin, cyclophosphamide), TAC (taxotere, AC), ABVD (Adriamycin, bleomycin, vinblastine, dacarbazine), BEACOPP (bleomycin, etoposide, Adriamycin, cyclophosphamide, vincristine, procarbazine, prednisone), BEP (bleomycin, etoposide, platinum agent (cisplatin (Platinol)), CAF (cyclophosphamide, Adriamycin, fluorouracil (5-FU)), CAV (cyclophosphamide, Adriamycin, vincristine), CHOP (cyclophosphamide, Adriamycin, vincristine, prednisone), ChIVPP/EVA (chlorambucil, vincristine, procarbazine, prednisone, etoposide, vinblastine, Adriamycin), CVAD/HyperCVAD (cyclophosphamide, vincristine, Adriamycin, dexamethasone), DT-PACE (dexamethasone, thalidomide, cisplatin or platinol, Adriamycin, cyclophosphamide, etoposide), FAC (5-fluorouracil, Adriamycin, cyclophosphamide), m-BACOD (methotrexate, bleomycin, adriamycin, cyclophosphamide, Oncovin (vincristine), dexamethasone), MACOP-B (methotrexate, leucovorin (folinic acid), adriamycin, cyclophosphamide, Oncovin (vincristine), prednisone, bleomycin), ProMACE-MOPP (methotrexate, Adriamycin, cyclophosphamide, etoposide+MOPP), ProMACE-CytaBOM (prednisone, Adriamycin, cyclophosphamide, etoposide, cytarabine, bleomycin, vincristine, methotrexate, leucovorin), VAD (vincristine, Adriamycin, dexamethasone), Regimen I (vincristine, Adriamycin, etoposide, cyclophosphamide) and VAPEC-B (vincristine, Adriamycin, prednisone, etoposide, cyclophosphamide, bleomycin).

Analogues of Doxorubicin for cancer chemotherapy include, but are not limited to, daunorubicin, 4-demethoxydaunorubicin (idarubicin), pirarubicin (DaunoXome), epirubicin, pegylated liposomal doxorubicin (Lipo-Dox®), antibody-conjugated liposomal doxorubicin (e.g. S5A8-Lipo-Dox), 4′-epidoxorubicin, ADI98, N-(5,5-Diacetoxypent-1-yl) doxorubicin, and Doxorubicin analogues 2-5, incorporating the following alkylating or latent alkylating substituents, R, on the 3′-position of the daunosamine sugar. 2, R—NHCOC.sub.6H.sub.4(p)SO.sub.2F; 3, R═NHCOCH.sub.2Br, 4, R═NHCOCH.sub.2Cl; 5, R═NHCON(NO)CH.sub.2CH.sub.2Cl. J Med Chem. 1991 February; 34(2):561-4.

As used herein, “Ibrutinib”, also known as PCI-32765, refers to a drug for the treatment of various types of hematopoietic related cancer. However, in one embodiment, the present invention contemplates the use of ibrutinib for non-hematopoietic related cancers, and in particular for breast cancer.

### DETAILED DESCRIPTION OF THE INVENTION

Embodiments disclosed herein include methods of treating cancer in a subject in need thereof, the method comprises administering an inhibitor of palmitoylation, wherein the inhibitor is an siRNA that corresponds to an exon sequence that code for the palmitoylated versions of BTK-C, ABL1, ABL2, and PDPK1. Embodiments disclosed herein include pharmaceutical compositions comprising a means for reducing the amount of palmitoylation sequences in BTK-C, ABL1, ABL2, and PDPK1 kinases in cancerous cells and a pharmaceutically acceptable carrier.

The embodiments of the present disclosure provide improved treatments that inhibit cancer cell growth but spare the immune system. Embodiments disclosed herein include, for example, targeting the palmitoylated isoforms of BTK-C, ABL1, ABL 2, and PDPK1, which are the predominant isoforms expressed in several solid tumor types with either small molecule inhibitors or suppressing the translation of the isoform through RNA interference (“RNAi”).

Embodiment disclosed herein included the following materials and methods: Cell lines MD-AMB-231, BT549, SUM149, SKBr3 and ϕNX-AMPHO were obtained from the American Type Culture Collection (ATCC). MD-AMB-231, SUM149, SKBr3, and Phoenix AMPHO cells were maintained in DMEM (Hyclone, Logan, UT) supplemented with 10% fetal bovine serum (FBS) (Hyclone). BT549 and LNCaP C4-2b cells were cultured in RPMI (Hyclone) with 10% FBS (Hyclone). All culture media contained 100 U/μl penicillin-streptomycin. All cell lines were authenticated in March 2016 by the SUNY-Albany Center for Functional Genomics Molecular Core Facility using a short tandem repeat method (Promega GenePrint 10 system, Madison WI). To minimize the influence of components contributed by bovine serum, all stimulation experiments were performed using 1% FBS unless otherwise indicated (18, 19). Cells were imaged as described. As localization was the key attribute being tested, images were normalized with respect to brightness and contrast. In growth and uptake experiments, differences between control and treatment conditions were evaluated using Student's t test for pairwise comparisons. Growth and glucose experiments were repeated 3 times. The average of at least three biological replicates were normalized to control and presented as mean+standard deviation.

Reporter construction and viral transgenesis: GFP fusion constructs were generated using a BTK pleckstrin homology domain fusion protein vector, PH-Btk-GFP (Addgene Plasmid #51463). BTK constructs (BTK-A R28C; BTK-C; BTK-C R28C; BTK-C C13A, C16A; BTK-C C13A,C16A,R28C) were generated in this plasmid using QuikChange site directed mutagenesis. The exon 1C sequence (FIG. 1B) was subcloned from the original cDNA isolate. To prevent internal translational initiation at the BTK-A ATG, this codon was changed to Ala in all BTK-C constructs. Production of either the 77 kDa BTK-A isoform or the 65 kDa p65BTK was not observed for BTK-C constructs. GFP reporters were subcloned into MARXIVpuro REV plasmid (22). Full-length mutant proteins were produced by subcloning into a BTK gene with a C-terminal Flag tag expressed from a MARXIVpuro REV vector. DNA sequence was confirmed by DNA sequence analysis for all constructs. ϕNX-Ampho cells were transfected with Flag-tagged versions of BTK as described. Empty vector was used as a control. A lentivirus-born shRNA targeting the chromosomal BTK 3′UTR TRCN0000000358 (Sigma-Aldrich, Saint Louis, MO) was used to silence endogenous BTK isoform expression such that each line predominantly expresses the wild-type or mutant version of the intended isoform. BTK acylation: HEK293 cells plated on 6 wells plates were transfected using X-tremeGENE HP transfection reagent (Sigma-Aldrich, Saint Louis, MO) with 1 μg of BTK-C, BTK-A, or BTK-C C13A, C16A with 2 μg of zDHHC5 plasmid. 24 hours after transfection, cells were incubated with 100 μM of palmitic acid alkyne (in DMEM with 1 mg/ml defatted BSA) for 4 h at 37° C. Cells were washed twice in PBS, then lysed on ice in 50 mM Tris HCl, pH 7.4 with 150 mM NaCl, 1 mM EDTA, 1% TRITON X-100 and protein inhibitors. Cell lysate supernatants were used for immunoprecipitation with Anti-Flag M2 affinity gel (Sigma-Aldrich, Saint Louis, MO) at 4° C. overnight. The affinity gel was washed with 0.5 ml of TBS three times and incubated with click chemistry reaction mixture (Click-&-Go Click Chemistry Reaction Buffer Kit, Click Chemistry Tools, Scottsdale, AZ) containing 20 μM biotin picolyl azide for 30 minutes at room temperature, separated by SDS PAGE and blotted.

Protein methods: Subcellular fractionation was performed using standard methods as described. Immunoblots were completed following standard protocols. Primary antibodies used were: P-Btk (Y223) (Cell Signaling Inc., Danvers, MA; #5082S), Flag Tag DYKDDDDK (D6W58) (Cell Signaling Inc., #14793S), Purified mouse anti-Btk (pY551)/ltk (pY511) (BD Biosciences, San Jose, CA; #558034), Histone H3 (Cell Signaling Inc., #9715S), Na/K ATPase (Epitomics, Burlingame, CA; #2047-X). Secondary antibodies: Anti-rabbit IgG, HRP-linked Antibody (Cell Signaling Inc., #7074S), Anti-mouse IgG, HRP-linked Antibody (Cell Signaling Inc., #7076S).

The alternative transcriptional isoform of Bruton's tyrosine kinase, BTK-C, is expressed in a wide variety of epithelial tumor types where it impacts apoptosis resistance, therapeutic escape, and glucose uptake. The initial exon in BTK-C encodes a 34 amino acid extension of the amino terminus of the canonical BTK-A isoform. Its function is unknown.

The cancer cell survival BTK-C isoform is phylogenetically conserved. BTK-C isoform, which has a transcriptional initiation site approximately 10 kb upstream of the BTK-A isoform (FIG. 1A) is the predominant form expressed in solid tumor cells in humans. Analysis of recent RNAseq data from the Genotype-Tissue Expression (GTEx) project confirms previous results indicating that the BTK-C isoform is expressed in relatively few tissues and at low levels under normal conditions (FIG. 1S). GTEx data show that the greatest expression of BTK-C is in testis, with lower levels of expression in both spleen and EBV-transformed lymphocytes. There is essentially no expression in other tissues.

An analysis of several species' genomes indicates that the exon 1C sequence (exon 1C is presented in the left column containing palmitoylation sites 13 and 16, respectively) is conserved in mammals (FIG. 1B). The 20 organisms in both primate and vertebrate list of the UCSC Genome Browser all display sequence conservation within this region. In each case, this includes encoded amino acid sequences of exon 1C as well as encoded amino acid sequences in exon 2 (exon 2 is presented in the right column of FIG. 1B, respectively) that are upstream of the start codon of the BTK-A isoform. In each organism, transcription initiation occurs divergently within a few hundred bases of the RPL36 ribosomal protein gene. The range of additional protein sequence is between 32 and 34 amino acids and is predicted to be unstructured. Importantly, analysis of the protein motifs found in these conserved sequences indicates that each contains potential palmitoylation sites (FIG. 1B). One or two cysteine residues are found in each exon 1C encoded amino acid sequence which score highly in the CSS-Palm palmitoylation motif prediction algorithm. Of note, some rodent sequences including mouse and rat share conserved sequences in the appropriate positions, however, a stop codon is found at the corresponding fourth amino acid of BTK-C and transcripts that map to this area are not annotated in mouse genome sequences. The mouse sequence was isolated by PCR and confirmed the presence of the stop codon at this position.

BTK-C expression in breast tumors: Analysis of expression of the BTK splice isoforms in tumor tissues in the TCGA database using the Tumor Splice Variant Database (TSV DB) indicates that virtually all tumor samples show expression of some BTK isoform within the tumor (FIG. 1C). BTK-A is widely expressed in the samples, although it is difficult to assess whether this is due to the presence of B-cell infiltrates in these tumors as BTK-A is expressed in hematopoietic cells at levels approximately 1000 times higher than those in solid tumor cells [not shown]. BTK-C expression is observed in 5-25% of tumors in a wide variety of cancers. This includes 14.4% (158/1100) of breast tumors, 12.5% (51/408) of bladder tumors, 23.5% (118/501) of lung squamous cell carcinomas and 5.8% (29/495) of prostate tumors.

Tumor samples from The Cancer Genome Atlas (TCGA) Firehose Legacy breast cancer database were parsed into two groups: tumors that expressed BTK-C(RNAseq reads mapped to exon1C>0) and those that do not express BTK-C (RNAseq reads mapped to exon1C=0). Analysis of attributes of the two groups indicated that tumors expressing BTK-C were more likely to be either ER or PR negative (FIG. 2S). There was, however, no statistically significant difference in overall survival between the groups. Analysis of transcripts differentially expressed in BTK-C expressing tumors are enriched in function-appropriate gene ontology biological process groups of ER sequestered calcium release, regulation of Toll-like receptor 2 signaling pathway and leukocyte aggregation. When compared to non-expressers, tumors expressing BTK-C show a statistically significant increase in a single protein species as measured by reverse-phase protein array: increased phosphorylation on threonine 346 of the hypoxia-induced, lipid metabolism gene, NDRG1(28-30) (FIG. 1D). Tumors that do not express BTK-C share clinical attributes having greater ER positivity or PR positivity which is also reflected in both transcriptomic and proteomic analysis. The top scoring gene ontology class of messages enriched in tumors not expressing BTK-C was “breast development and signaling” which includes transcripts for the estrogen receptor (ESR1), androgen receptor (AR), progesterone receptor (PGR) and the EGF family member ERBB4, among others. Many of these proteins were also found to exhibit statistically significant higher levels of expression in proteomic analysis of tumors not expressing BTK-C including the estrogen receptor, the androgen receptor, and ERBB2 among others (FIG. 1D). Although expression of BTK-C does not track with a specific breast cancer subtype, taken together, the results indicate that tumors expressing BTK-C are likely to have reduced levels of canonical breast cancer signaling receptor proteins.

The BTK-C isoform but not BTK-A is palmitoylated: Analysis of the predicted BTK-C sequence with motif identification algorithms indicated that cysteine residues 13 and 16 in the BTK-C 34 amino acid N-terminal extension are highly scoring predicted Type II palmitoylation residues (FIG. 1B). Conserved motifs for N-glycosylation or N-myristoylation were not found. Palmitoylation is a dynamic post-translational modification of proteins in which palmitate is covalently bonded to cysteine residues of the mature protein. The linkage occurs through a reversible thioester bond catalyzed by any of a large family of protein acyl transferases the substrate specificities of which are at present poorly understood. Palmitoylation was originally thought to be a mechanism for targeting to lipid rafts in the plasma membrane. It is now more generally considered to play an important role in subcellular trafficking of substrates many of which function in signal transduction. So that the functional significance of the predicted palmitoylation sites at Cys13 and Cys16 within the N terminal domain of human BTK-C could be determined, these sites were mutated to alanine residues using in vitro mutagenesis (FIG. 2A).

To determine whether the predicted palmitoylation sites of BTK-C are acylated, Flag-tagged versions of BTK-C wt, BTK-A wt, or BTK-C: C13A, C16A were transfected (1 μg each) and expressed in HEK293 cells (FIG. 2B) with 2 μg of a plasmid expressing the plasma membrane-localized palmitoyl transferase, zDHHC5. The cells were subjected to acyl incorporation assays (FIG. 2C) in which modification with an acyl analogue is evidenced using click chemistry modification after FLAG based immunoprecipitation. Analogues of palmitic acid, 17-octadecynoic acid (17-ODYA, 13266 Cayman Chemical, Ann Arbor, Michigan), palmitoleic acid (25362 Cayman Chemical, Ann Arbor, Michigan), oleic acid (9002078 Cayman Chemical, Ann Arbor, Michigan) and arachidonic acid (10538 Cayman Chemical, Ann Arbor, Michigan) were tested for incorporation. As shown in FIG. 2D, label is increased in BTK-C but not BTK-A, or the BTK-C C13A, C16A double mutant. We failed to observe incorporation of palmitoleic acid, oleic acid and arachidonic acid (not shown). We were also unable to see incorporation without co-expression of the palmitoyl transferase; nevertheless, these experiments confirm that the N terminal domain of BTK-C is a substrate for palmitoylation.

Palmitoylation affects BTK-C cellular localization: BTK is established as soluble kinase that is recruited to the membrane by PI3K activity. Fusions of the pleckstrin homology domain of BTK (amino acids 1-177) with GFP are widely used to monitor PIP₃ levels in cells. We modified this construct to incorporate the exon 1C sequence of BTK-C and several site-directed mutations (FIG. 3A). Each construct was subcloned into a PuroMaRX IV-based retrovirus and transduced into a variety of cell lines. Mutation of either cysteine residue by itself in the exon 1C sequence is largely without effect on subcellular localization of BTK-C(FIG. 3S). Mutation of both cysteine 13 and cysteine 16, however, caused the reporter construct to localize in the cell interior (FIG. 3B).

Both BTK-A GFP and BTK-C GFP fusions exhibit significant plasma membrane localization; however, images focused on the mid-depth of cancer cells show that the BTK-A isoform is largely localized within the nucleus whereas the BTK-C isoform has a primarily perinuclear distribution (FIG. 3B). The nuclear localization of BTK-A in solid tumor cells is not surprising. The BTK-A isoform has been shown to shuttle between the cytoplasm and nucleus in B cells. During this process, export from the nucleus requires association with the LIAR protein. This interaction occurs within the SH3 domain which is missing in these pleckstrin homology domain reporters but is retained in the full-length constructs. Analysis of BTK-GFP fusion signal in the cells with CellProfiler image quantification analysis showed that approximately 60% more BTK-A signal is localized to the nucleus compared to BTK-C. Fractionation of cells expressing Flag-tagged, full-length versions of BTK-A and BTK-C and subsequent immunoblotting of nuclear and cellular membranes shows that BTK-C is more abundant in cellular membranes compared to BTK-A which is found in higher amounts in the nucleus (FIG. 3C).

Mutations in the palmitoylation sequences at the cysteine residues in BTK-C (C13A, C16A) substantially decreased plasma membrane localization of BTK-C-GFP. Additionally, mutation of the amino acid corresponding to arginine 28 of the BTK-A sequence to cysteine as occurs in mice also abolished membrane localization. This position in the pleckstrin homology domain interacts with PIP₃ during activation. The same R28C mutation in BTK-C causes it to fail to localize to the membrane indicating that BTK-C requires both palmitoylation and PI3K signaling to associate with the membrane. A common downstream effect of BTK activation due to B cell receptor engagement is actin polymerization. We observed that considerable BTK-A and BTK-C reporter localization is juxtaposed to actin filaments in the breast cells (FIG. 3D).

Regulation of BTK-C localization and activity by PIP₃ levels: Activation of BTK-A in B cells has been extensively studied. Adsorption of BTK-A to the B cell plasma membrane is driven by binding to PIP₃ which opens the BTK monomer conformation allowing dimerization, auto-activation (Y223 phosphorylation) and transactivation (Y551 phosphorylation). Results from several lines of experimentation indicate that the additional sequence at the BTK-C amino terminus does not alter the regulation of the kinase by PIP₃. The positively charged R28 residue in the PH domain has been shown to contact the 5′-PO₄ of PIP₃ and mutation of this residue makes the kinase insensitive to PI3K signaling. Mutation of the analogous arginine in BTK-C(R62 in BTK-C) causes it to fail to localize to the membrane (FIG. 3S). Both BTK-A and BTK-C isoforms exhibit significant localization in the plasma membrane of cells that harbor mutations in PTEN, the phosphatase that removes the D3 phosphate and reduces PIP₃ levels (FIGS. 4A and 4B). For example, MDA-MB-231 cells which are PTEN⁺ display less plasma membrane staining when compared to SUM149 cells which are PTEN. In these cells, BTK-A also shows increased plasma membrane localization but to a lesser degree than BTK-C. Since there are a limited number of PTEN-cell lines, we also used pharmacological inhibition with IPI-145 (Selleckchem, Houston, TX) (44), Duvelisib, a highly selective PI3K δ/γ inhibitor that decreases the membrane signal of both BTK-A and BTK-C GFP fusions. In breast cells, this is accompanied by significant toxicity (not shown). However, there is less toxicity in the PTEN-LNCaP C42b prostate cancer cell line that expresses BTK-C, where IPI-145 inhibition dramatically decreases the degree of membrane localization observed in controls (FIG. 4B). Similarly, expressing wild-type PTEN in the PTEN⁻ LNCaP C42b prostate cancer cell line decreases BTK-C activation whereas expressing dominant negative mutants does not (FIG. 4D). This PIP₃-dependent activation may occur through BTK-C dimer formation through juxtaposed membrane-bound PH domains as occurs with the BTK-A. Using an assay developed by Chung et al., co-transfecting BTK-C with the BTK-C-GFP construct which contains the PH domain but is missing the kinase domain reduces Y551 phosphorylation compared with vector or BTK-C only (FIG. 4D). This result is consistent with its interfering with dimer formation as has been shown for similar BTK kinase domain constructs in other transfection-based studies.

We assessed the in vivo activity of the BTK isoforms by co-transfecting wild-type and mutant constructs into HEK293 cells (FIG. 2 and FIG. 4S). BTK-A and BTK-C are both activated in the cells as judged by tyrosine phosphorylation at Y223 and Y551. As shown in FIG. 4C, BTK-C: R28C (50% BTK-C wt), BTK-C: C13A, C16A (33% BTK-C wt) and BTK-C: C13A, C16A, R28C (14% BTK-C wt) proteins exhibit lower Y551 phosphorylation levels suggesting that BTK-C activation by other activating kinases may depend on its correct localization which requires both palmitoylation and PIP₃ binding ability. BTK transactivation was less impacted by these mutations with only the BTK-C: C13A, C16A (33% BTK-C wt). Expression of BTK-C with a mutation analogous to BTK-A E41K (E75K in BTK-C numbering) which increases the positive charge in the PH domain and has been shown to increase membrane recruitment and activation (42), does not exhibit appreciably increased activation in these cells. PLCγ2, an established BTK effector in hematopoietic cells, is expressed in a variety of epithelial cancer cells (46). As shown in FIG. 5B, overexpression of either isoform increases phosphorylation of PLCγ2 on tyrosine 759. Interestingly, mutation of only the palmitoylation residues does not impact PLCγ2 phosphorylation appreciably, although mutation of both palmitoylation and PIPs binding site does.

BTK-C activity in cancer cells in vitro: Previous studies have shown that both BTK-A and BTK-C are active in proliferating breast and prostate cancer lines under normal conditions in vitro. To determine whether the two isoforms of BTK responded differently to stimulation, we examined activation in response to insulin stimulation under reduced serum conditions. Insulin is a common activator of PI3K in cancer cells and has been shown to cause activation of BTK in other cell types. Insulin stimulation increases Y551 phosphorylation, reflecting activation by other kinases, two-fold in BTK-A, however considerably less (˜24%) in BTK-C. Transactivation, reflecting phosphorylation of BTK by its dimerized partner, is largely unchanged in BTK-A and BTK-C under these conditions (FIG. 5A). Other ligands including EGF, NRG and RANKL have a similar impact (not shown) increasing activating phosphorylation levels above unstimulated levels only modestly. These results are in accord with the original observations that the kinase is active in proliferating cancer cells and suggests that further work is needed to identify the pathway or pathways responsible for activation in a given cell type. Nevertheless, even without exogenous stimulation, the baseline BTK activity has functional consequences that correlate with the palmitoylation status of BTK in breast cancer cells. We observe a statistically significant increase in proliferation, which was reproduced in multiple experiments, of 17.6% in PTEN⁺ MDA-MB-231 cells expressing BTK-C (+endogenous BTK-targeting shRNA) compared to BTK-A (+endogenous BTK-targeting shRNA) (FIG. 5C). Effects of the expression of the BTK-C C13A, C16A double mutant does not differ appreciably from the BTK-A isoform (data not shown). Cell proliferation rates of PTEN-SUM149 cells exhibited similar rates of growth irrespective of the isoform expressed. Since our previous work has shown that BTK-C activity is correlated with glucose uptake in breast cancer cells, both lines expressing full length BTK-A and BTK-C wild-type constructs were tested for effects on glucose uptake by assaying 2-NBDG fluorescence. The results show that in MDA-MB-231 cells, there is a reproducible 25% increase in glucose uptake in those cells expressing BTK-C when compared to BTK-A (FIG. 5D). Similar to the impacts on proliferation, there are no significant differences in glucose uptake activity between BTK-A and BTK-C constructs in PTEN-SUM149 cells. We surmise that the lack of significant differences between the two isoforms when expressed in the PTEN-cell is due to constitutive activation of the non-palmitoylated form.

PI3K effector kinase isoform generation through alternative start site selection: Results of these experiments on BTK-C indicate that an alternative amino terminus can impact kinase localization and function. BTK is one of 5 TEC family kinases encoded in the human genome. Although the TXK kinase does not contain a bona fide PH domain, it is the most highly related TEC kinase to BTK. Previous studies have established that the 5′ end of the TXK mRNA possesses two alternative translational start sites, one of which encodes a run of cysteine residues that is palmitoylated and produces a predominantly perinuclear localized product. Each of the other TEC kinases has a single 5′ end version and none are predicted to be palmitoylated.

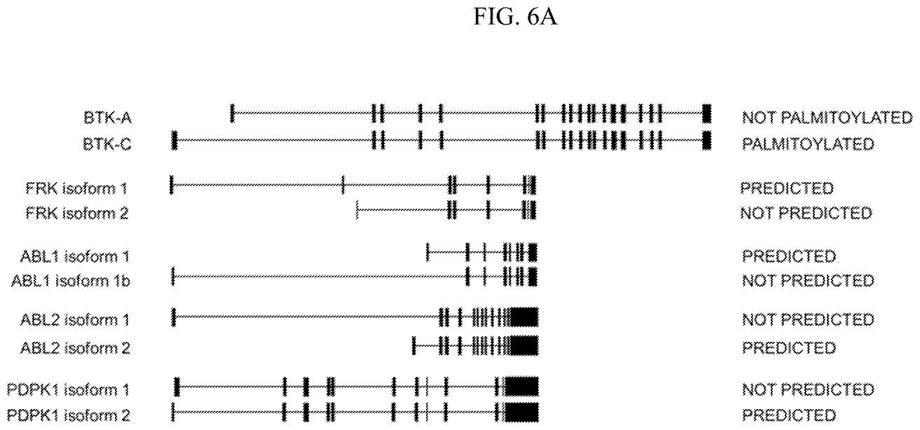

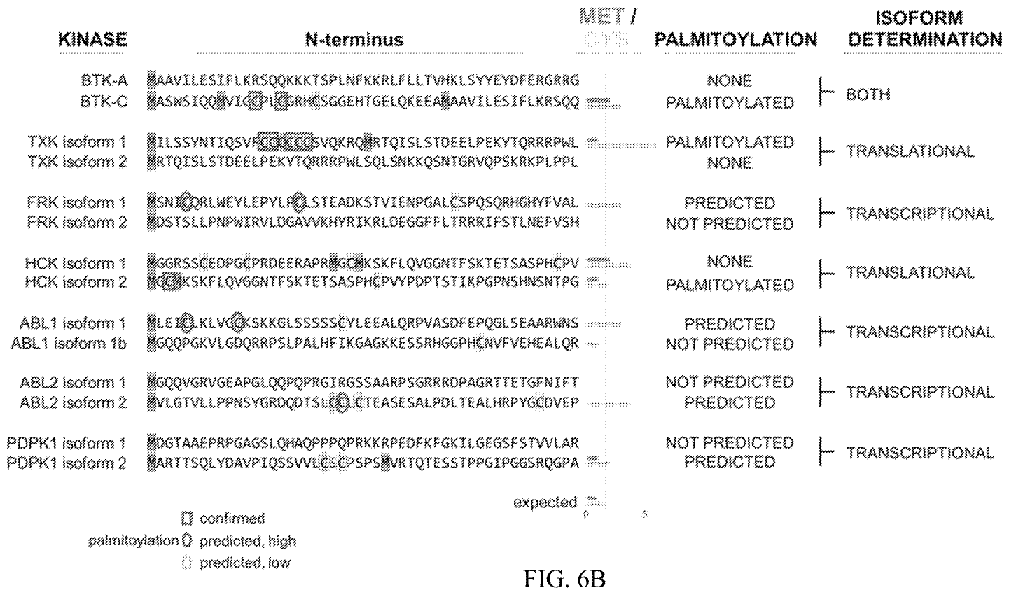

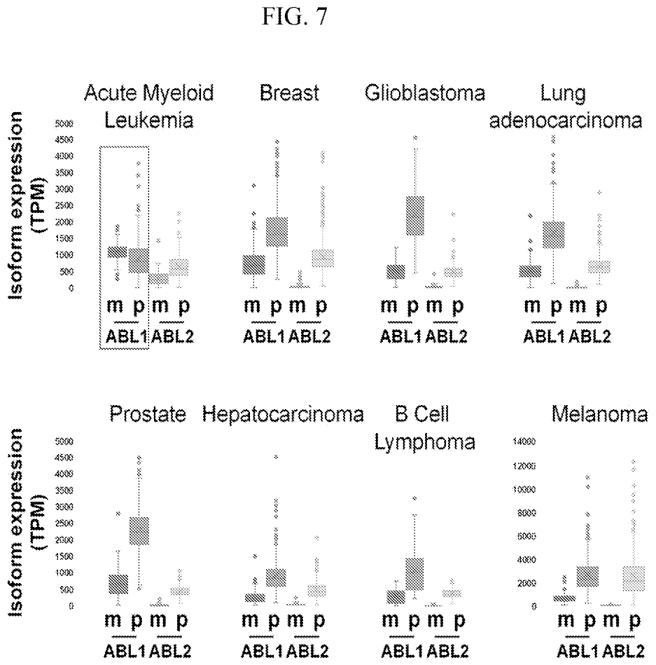

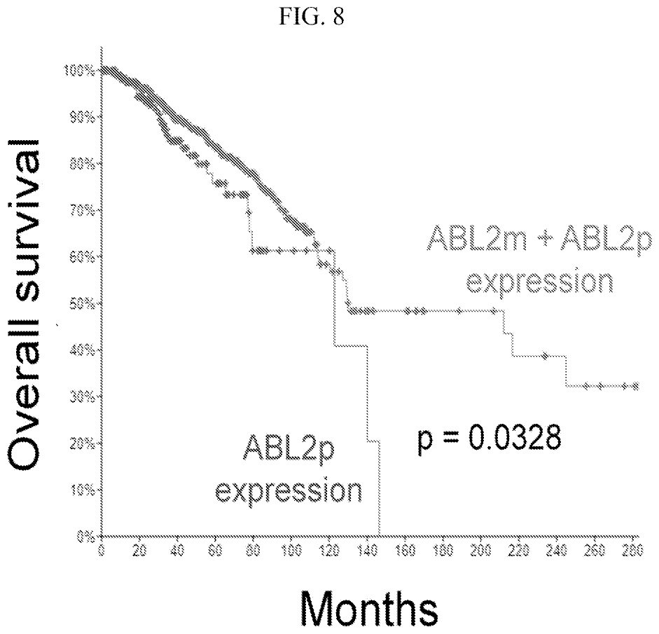

To extend the observations on the TEC kinases, we determined how many kinases in the human genome transducing signals at the plasma membrane, have the potential to encode either palmitoylated or non-palmitoylated versions. An analysis of all soluble, non-receptor tyrosine kinases and all PH domain-containing serine/threonine kinases (FIG. 5S) showed that 4 others have alternative first exons: FRK, ABL1, ABL2 and PDPK1 (FIG. 6A); these encode either palmitoylated or non-palmitoylated amino termini analogous to BTK-A and BTK-C(FIG. 6B). Three other kinases (JAK1, JAK2 and UFO) are similar to TXK (50), having the potential for either palmitoylated or non-palmitoylated amino termini determined by translational initiation site choice (FIG. 5S). Additionally, HCK, whose expression involves both alternative transcriptional and translational start sites and myristoylation, has previously been demonstrated to have both palmitoylated and non-palmitoylated versions (FIG. 6B).

Analysis of coding regions for these 10 kinases shows that those isoforms with confirmed or predicted palmitoylation sites are enriched in cysteine residues near the amino terminus. Those with the potential for alternative translational start sites are also enriched in methionine, with most having at least one methionine occurring after the palmitoylation site, as occurs in BTK and TXK to produce a non-palmitoylated version. The predicted and established non-palmitoylated isoform sequences of these kinases are depleted of both these amino acids over the first 50 amino acids (FIG. 6B and FIG. 5S). In this way, amino terminus choice appears to be a binary switch that impacts the localization and activity of these kinases. These kinases, which we classify as alternative N-terminal (ANT) kinases, include several tyrosine kinases as well as PDPK1, a major PI3K-effector serine threonine kinase. Determining the function of these altered isoforms will require additional investigation. However, it underscores the notion that alternative amino termini, as occurs with BTK, is a common feature of kinases with important roles in cancer cell signaling.

In FIG. 1S Genotype-Tissue Expression (GTEx) project expression of the BTA-A and BTK-C variants. The BTK-C isoform is expressed in relatively few tissues and at low levels under normal conditions. The greatest expression of BTK-C is in testis, with lower levels of expression in both spleen and EBV-transformed lymphocytes. There is essentially no expression in other tissues. BTK-A has highest expression in peripheral lymph tissues.

In FIG. 2S tumor samples from the TCGA Firehose Legacy breast cancer database were parsed into two groups: tumors that express BTK-C(RNAseq reads mapped to exon1C>0) and those that do not express BTK-C(RNAseq reads mapped to exon1C=0). Statistically significant differences were found in the clinical attributes of these groups. In excess of 36% of tumors expressing BTK-C were estrogen receptor (ER) negative compared to only 20.3% of tumors not expressing BTK-C (p-value=8.724×10⁻⁵; q-value=2.982×10⁻³). Similarly, 41% of tumors expressing BTK-C were progesterone receptor (PR) negative compared to 31% of tumors not expressing BTK-C (p-value=9.620×10⁻⁵; q-value=2.982×10⁻³). Although BTK-C expressing tumors were more likely than BTK-C not expressing tumors to be either ER or PR negative, a greater proportion were positive for these markers.

In FIG. 3S the expression of site-directed mutations after transient transfection into HEK 293 cells is presented. BTK-A exhibits both cytoplasmic and plasma membrane staining. Wild type BTK-C displays plasma membrane localization (left panel). As occurs with BTK-A [1-3] (not shown), mutation of the arginine analogous to R28 in BTK-A (R62 in BTK-C), which removes a positive charge that contacts the 5′-PO₄ of PIP₃ [1, 2], causes it to fail to localize to the membrane (left panel). Mutation of either cysteine residue in exon 1C sequence is largely without effect on subcellular localization of BTK-C (right panel). Mutation of both cysteine 13 and cysteine 16, however, causes the reporter construct to localize in the cell interior. These data indicate that BTK plasma membrane localization in HEK293 cells requires both palmitoylation and PIP₃ interaction.

FIG. 4S presents expression of full length BTK constructs in MDA-MB-231 cells. Full immunoblots are shown.

FIG. 5S presents an analysis of all soluble tyrosine kinases and all PH domain-containing serine/threonine kinase genes for the presence of alternative encoded amino termini. Kinase genes were examined in human genome release Genome Reference Consortium Human GRCh38.p12. Kinases with alternative translational initiation sites are from the Uniprot database. Sequences are arranged using Jalview. Kinases with a single transcriptional start site and initial exon are shown once on the list. Kinases with two potential transcriptional start sites and first exons appear twice. Kinases with established alternative translational initiation sites have more than one methionine start depicted (green boxed). The first 50 amino acids of each first exon were analyzed for the presence of methionine and cysteine residues. Based on average amino acid usage in humans, 0.9 methionine and 1.65 cysteine residues would be expected in a random 50 amino acid sequence. The initial ATG is not counted toward the total. Amino acid sequences were also tested for the presence of potentially acylated residues using the palmitoylation site CSS-Palm (http://csspalm.biocuckoo.org/). Canonical and established alternative translational initiation sites are boxed in green. Confirmed palmitoylation sites are circled; predicted palmitoylation sites are boxed in orange. In total, 10 kinases in the human genome, BTK, FRK, HCK, ABL1, ABL2, PDPK1, TXK, JAK1, JAK2 and UFO are alternative N-terminal (ANT) class kinases having the potential to produce either acylated or non-acylated amino termini as occurs with BTK-A/BTK-C. In some instances, this is due to the use of an alternative transcriptional start site, while in others the choice of N-terminus is determined by translational initiation. BTK, FRK, HCK, ABL1, ABL2 and PDPK1 all have alternative first exons encoding either palmitoylated or non-palmitoylated amino termini (established or predicted). BTK, HCK, TXK, JAK1, JAK2 and UFO have encoded methionine residues near the 5′ end of the gene that if chosen as the translational start would produce a predicted non-palmitoylated product. Aside from BTK, TXK and HCK there is currently no evidence to indicate that translational initiation occurs to produce alternative amino termini for these kinases.

Our previous findings showed that the BTK-C isoform is expressed in human breast and prostate cancer cells and that it plays a crucial role in epithelial cancer cell survival. We have found that the 34 amino acid extension of BTK-C contains a phylogenetically conserved palmitoylation site that localizes the kinase to membranes and has context-dependent functional consequences. Although originally identified as the predominant isoform expressed in solid tumor cells, our work shows that BTK-C is not expressed at significant levels in most normal tissues or cells. Its expression profile and its role as a critical survival factor for breast and other solid tumor cells makes BTK-C a specific, druggable target for treating a wide range of epithelial cancers.

We observed that the BTK-C isoform is palmitoylated but only when the plasma membrane-localized palmitoyltransferase zDHHC5 was also overexpressed. This reaction appears to be inefficient in these experiments (FIG. 2) which might reflect another, non-tested acyl group as the primary possible modification. Nevertheless, mutation of the acylated cysteines C13 and C16 had obvious effects on the subcellular localization of the BTK kinase in cells. BTK-C displays significant perinuclear localization as occurs with several palmitoylated family kinases, such as SRC, LYN and YES, as well as the TEC family TXK. Although palmitoylation is important to the activity of these kinases, the impact that palmitoylation has on the dynamics of signaling is poorly understood in these instances and complicated somewhat by the fact that these enzymes are also myristoylated. Association of BTK-C with the membrane required either C13 or C16 and a functional PH domain for membrane association and impacted kinase activation (FIG. 5). These results may indicate that BTK-C has a somewhat different mechanism of activation from BTK-A which is activated at the plasma membrane through PIP₃ interaction alone.

Both the palmitoylated and non-palmitoylated isoforms responded to PI3K signaling in increased plasma membrane association. However, molecular mechanisms responsible for phosphorylation-based activation of the kinases (FIG. 4C and FIG. 5A) in solid tumor cells may be less straightforward than those involved in activation by B cell receptor signaling. Expression of full-length BTK-C improved glucose uptake and proliferation of triple negative MDA-MB-231 cells but did not have a significant effect on PTEN-SUM149 cells. We surmise that increased localization of BTK-A in the PTEN-SUM149 cells reduces the difference in activity of the isoforms. In these cells and in general, the lack of dramatic differences between the isoforms may reflect the fact that the palmitoylated isoform exists primarily as a targeting mechanism in cells that have larger surface areas, receptor repertoires, and cytoplasmic volumes as well as more developed endomembrane systems. B cells, which express the BTK-A isoform, are significantly different from cells of epithelial origin that express BTK-C in this respect.

The arrangement of the BTK gene with alternative 5′ exons encoding alternative amino termini, appears to be an underappreciated paradigm in soluble kinases in mammals. 10 kinases possess encoded, phylogenetically conserved, alternative N termini with potential impacts on tumor biology and progression. For example, BTK-C expression in breast tumors is correlated with lower protein expression of common breast cancer signaling receptors, consistent with the notion that expression of BTK-C or other palmitoylated kinases in solid tumors increases the level of PI3K effector activity irrespective of signaling inputs. There are other possible interpretations and although additional experimentation is required, this explanation would fit with the observed effects of BTK-C expression in solid tumors including increased survival, apoptosis resistance, and chemotherapeutic escape.

For other alternative kinases besides BTK, similar attributes exist which present therapeutic opportunities especially for solid tumors. We know that the palmitoylated isoforms of ABL1 and ABL 2 are the predominant isoforms expressed in several solid tumor types (FIG. 7). Similar data also exist for PDPK1 (not shown). ABL1 and ABL2 are well-established cancer targets. One of these kinases, ABL2, was identified in a screen that we performed as the top potential target for killing breast cancer cells. Although ABL1 and ABL2 already have several drugs that target them, the non-palmitoylated form, which is myristoylated for both ABL1 and ABL2, is the version for which inhibitors have been designed. This likely originally stems from the fact that mutant ABL1 is a cause of acute myeloid leukemia and the predominant isoform expressed in this type of cancer is the myristoylated version (FIG. 7, red box). As shown in FIG. 1, however, in several other tumor types including breast, glioblastoma, lung adenocarcinoma, prostate cancer, hepatocarcinoma, B cell lymphoma, and melanoma, the palmitoylated versions of both ABL1 and ABL 2 are the predominant isoform expressed in cancer cells. Although most of the current ABL1 or ABL2 inhibitors are likely to inhibit both isoforms, part of the value of this work is that we can see how to target the most relevant form of each kinase.

Specifically targeting palmitoylated isoforms of these kinases represents a potential precision-based approach to cancer therapy. For example, an analysis of breast tumors, shows that patients whose tumors express the palmitoylated isoform of ABL2 but not the non-palmitoylated form have a statistically significant poorer outcome than patients whose tumors also express the ABL2 non-palmitoylated isoform FIG. 8.

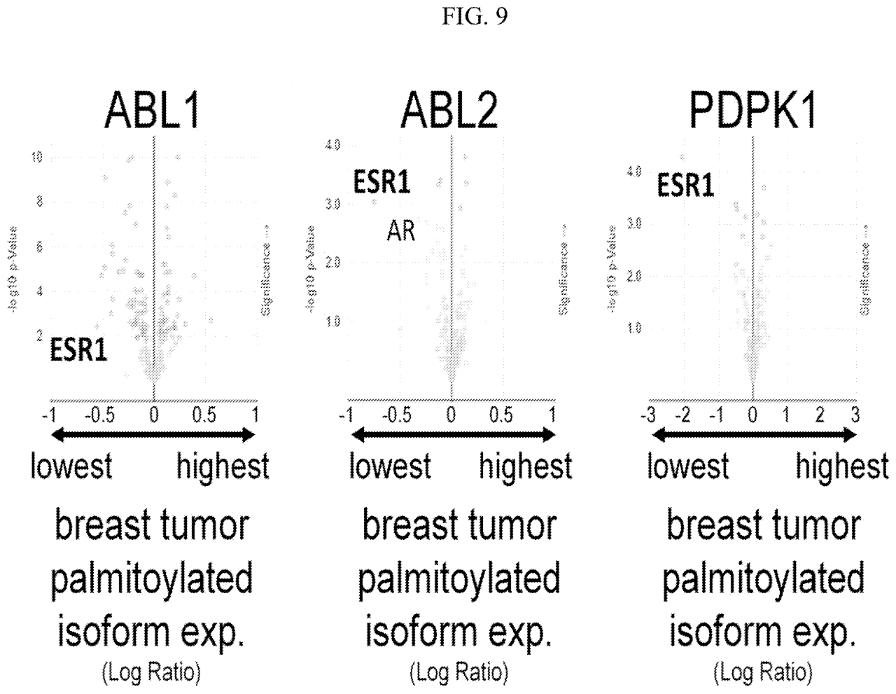

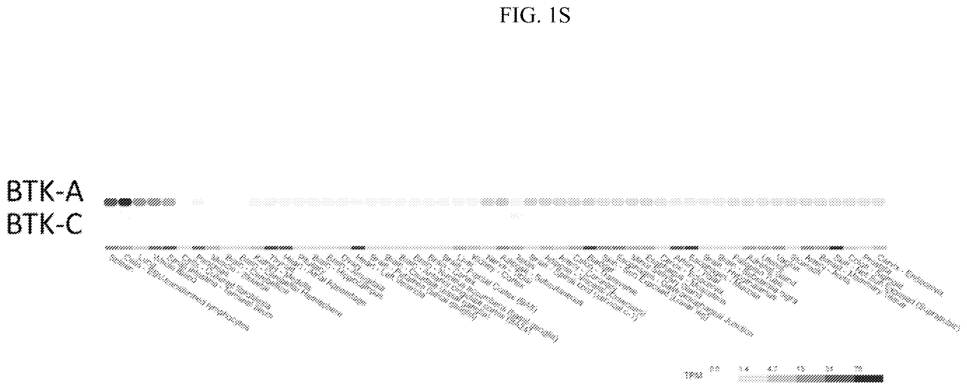

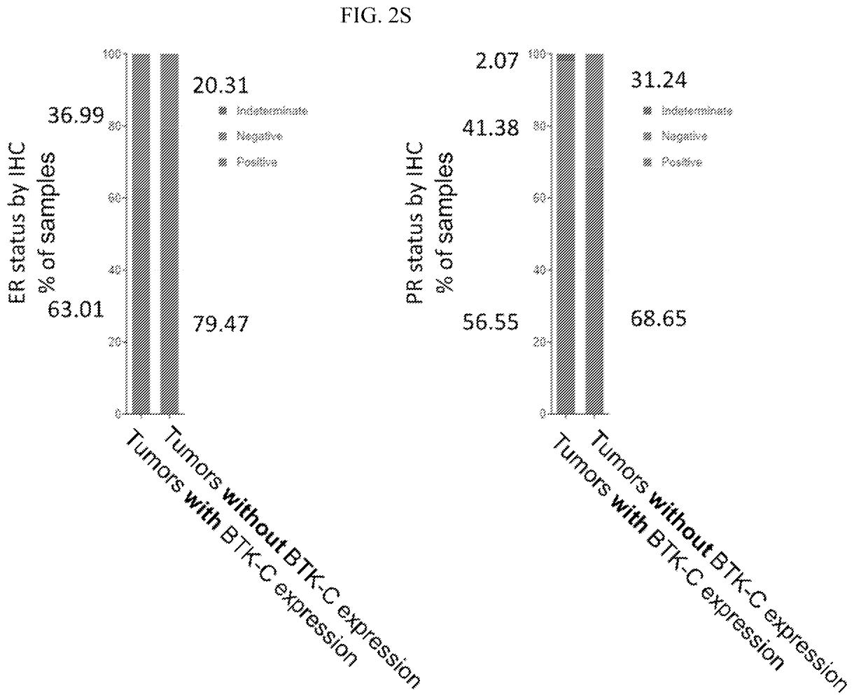

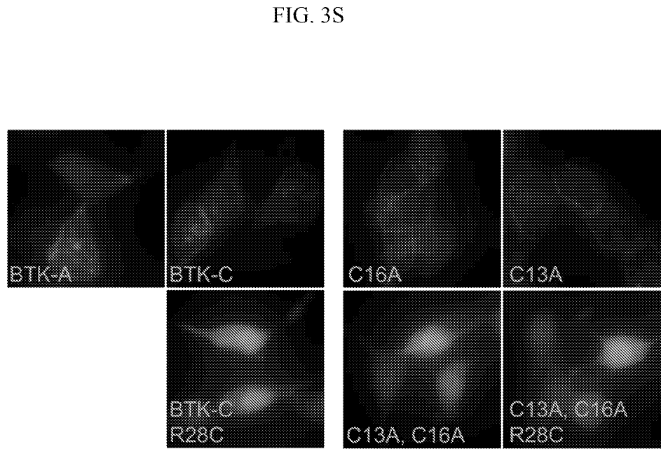

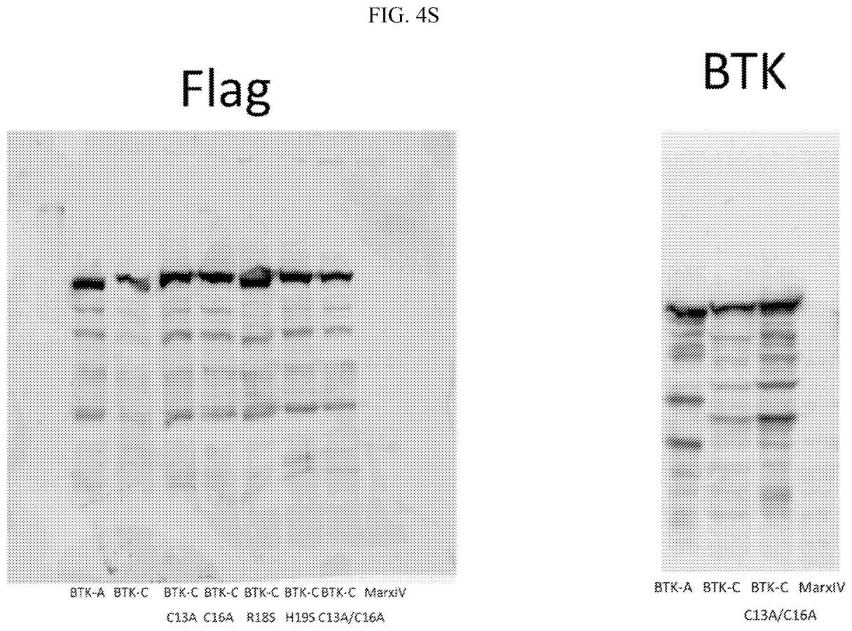

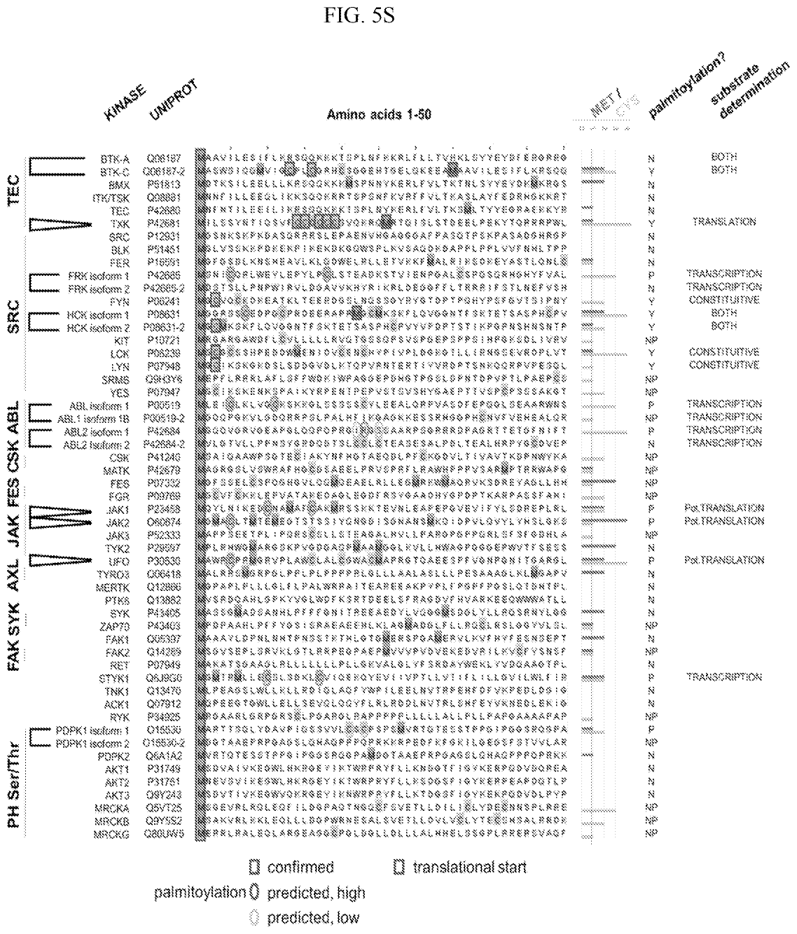

Targeting the palmitoylated isoforms may have value in that these versions appear to be expressed in cancer cells with lower levels of expression of common drug targets. For example, breast tumors in the Cancer Genome Atlas (TCGA) database that possess the highest quintile of ABL1, ABL2, or PDPK1 expression are less likely to express the estrogen receptor FIG. 9. Similar expression patterns are seen with BTK-C and detailed in FIG. 1. Together this suggests that drugs targeting these palmitoylated isoforms may be useful since they are viable approaches in tumors that have low level of expression of commonly targeted pro tumor receptors. For example, breast tumors that do not express BTK-C have clinical attributes of greater ER positivity or PR positivity express statistically significant higher levels of the estrogen receptor, the androgen receptor, EGF, and ERBB2 among others. Although expression of BTK-C is not completely correlated with a specific breast cancer subtype or worse outcome, taken together, the results indicate that tumors expressing BTK-C are likely to have reduced levels of canonical breast cancer signaling receptor proteins that are usually targeted with current therapies.

Potential composition of matter: Of the 10 Alternative N terminal kinases, half have the alternative version specified by an additional transcriptional initiation site and alternative first exon. These include ABL1, ABL2, and PDPK1. Since the palmitoylated versions of these proteins are more highly expressed in solid tumors, RNA molecules that target the first exons that encode the palmitoylation sites could be used to reduce the activity of these kinases in tumors. Based on the activities of these proteins, this is likely to be important in counteracting cancer cell survival, proliferation, and aggressiveness.

Because, like BTK, the palmitoylated version is the isoform that is predominantly expressed in solid tumors, targeting the palmitoylated versions of ABL1, ABL2, and PDPK1 is likely to have therapeutic benefit especially in solid tumor cells. Since the palmitoylated isoforms are produced through the use of an alternative transcriptional start site and alternative exon one, the palmitoylated isoforms can be targeted at the RNA level using RNA interference methodologies. A number of siRNA-based therapies have shown promise in recent clinical trials as anti-cancer agents following systemic administration. SiRNAs that correspond to the exon one sequences that code for the palmitoylated versions of ABL1, ABL2, and PDPK1 can provide a means for specifically targeting these enzymes in solid tumors. Embodiments disclosed herein contemplate the use of interfering double stranded RNAs that are at least partially complementary to the palmitoylation site encoding sequences shown that inhibit expression of the acylated proteins encoded.

Identification of palmitoylation sites and their potential as sites for drug interaction: It is understood that BTK-C has a functional part that is different from the canonical isoform (BTK-A). The BTK-C alternative isoform is palmitoylated, which as described above, which has been found to be the case for several other kinases. The most likely conformation rendered by AlphaFold 2.0 of the 34 amino acid extension unique to BTK-C is largely unstructured with a short alpha helical sequence predicted near the junction with the PH domain (FIG. 4E). The palmitoylated residues C13 and C16 lie on either side of a proline residue at position 14 which introduces a turn in the extension. We hypothesize that this “mooring whip” structure allows the protein to be tethered to the membrane yet allows (via glycine residues, G22, G23 and G27) for PIP₃-dependent PH domain association on the inner surface of the membrane.

The relevant palmitoylation-site-containing exon 1 sequences for certain of these kinases in the human genome (ABL1, ABL2, and PDPK1) and corresponding exemplary siRNA (“sense strand”) sequences are presented below in Table 1.

TABLE 1
ABL1 Palmitoylated Exon 1
SEQ ID NO. 1
GTTAACAGGCGCGTCCCGGCCAGGCGGAGACGCGGCCGCGGCCATGGG
GGGGCGCGGGCGCGCGGGGCGGCGGTGAGGGGGGCTGGCGGGGCCGGG
GGCGCCGGGGGGGCGCGCGGGCCGAGCCGGGCCTGAGCCGGGCCCGCG
GACCGAGCTGGGAGAGGGGTTCCGGCCCCCGACGTGCTGGCGCGGGAA
AATGTTGGAGATCTGCCTGAAGCTGGTGGGCTGCAAATCCAAGAAGGG
GCTGTCCTCGTCCTCCAGCTGTTATCTGGAAG
Potential siRNA sequences:
SEQ ID NO. 2
5′-GA AAA UGU UGG AGA UCU GUU-3′
SEQ ID NO. 3
5′-GCG GGA AAA UGU UGG AGA UUU-3′
SEQ ID NO. 4
5′-GGG AAA AUG UUG GAG AUC UUU-3′
SEQ ID NO. 5
5′-GAA GCU GGU GGG CUG CAA AUU-3′
SEQ ID NO. 6
5′-AAG CUG GUG GGC UGC AAA UUU-3′
ABL2 Palmitoylated Exon 1
SEQ ID NO. 7
ACAGTTTAACTTTTGTGCTTCTGGGCAGAGGTATGGTCCTTGGGACAG
TTCTCCTTCCACCTAATAGTTATGGCAGAGATCAGGACACTTCACTTT
GCTGCCTGTGCACTGAGGCCTCAGAATCTGCTCTACCCGACTTAACAG
Potential siRNA sequences:
SEQ ID NO. 8
5′-GGG CAG AGG UAU GGU CCU UUU-3′
SEQ ID NO. 9
5′-ACC UAA UAG UUA UGG CAG AUU-3′
SEQ ID NO. 10
5′-GAU CAG GAC ACU UCA CUU UUU-3′
SEQ ID NO. 11
5′-GCA GAG AUC AGG ACA CUU CUU-3′
SEQ ID NO. 12
5′-UGG CAG AGA UCA GGA CAC UUU-3′
PDPK1 Palmitoylated Exon 1
SEQ ID NO. 13
GGCGGGCGCAGGATGAGGGCGGCCATTGCTGGGGCTCCGCTTCGGGGA
GGAGGACGCTGAGGAGGCGCCGAGCCGCGCAGCGCTGCGGGGGAGGCG
CCCGCGCCGACGCGGGGCCCATGGCCAGGACCACCAGCCAGCTG
Potential siRNA sequences:
SEQ ID NO. 14
5′-GGG AGG AGG ACG CUG AGG AUU-3′
SEQ ID NO. 15
5′-GGA GGA GGA CGC UGA GGA GUU-3′
SEQ ID NO. 16
5′-AGG AGG ACG CUG AGG AGG CUU-3′
SEQ ID NO. 17
5′-UCG GGG AGG AGG ACG CUG AUU-3′
SEQ ID NO. 18
5′-GCU UCG GGG AGG AGG ACG CUU-3′
SEQ ID NO. 19
5′-GGC CAG GAC CAC CAG CCA GUU-3′

Homo sapiens Bruton tyrosine kinase (BTK), transcript variant 3, mRNA, including BTK-C protein-coding sequence (nucleotides 244 to 2329 in this sequence; note that amino acid residues 13 and 16 in the corresponding protein sequence (full 693 amino acid sequence provided as SEQ ID NO. 21) are the sites of palmitoylation), NCBI Reference Sequence: NM_001287344.2 (incorporated herein by reference) are presented in Table 2. (available at ncbi.nlm.nih.gov/nuccore/NM_001287344.2)

TABLE 2
SEQ ID NO. 20

| | |
|---|---|
| 1 | ttcgtcactt tatctctttt ggtggactct gctacgtagt ggcgttcagt gaagggagca |
| 61 | gtgtttttcc cagatcctct ggcctccccg tccccgaggg aagccaggac tagggtcgaa |
| 121 | tgaaggggtc ctccacctcc acgttccatt cctgttccac ctcaaggtca ctgggaacac |
| 181 | ctttcgcagc aaactgctaa ttcaatgaag acctggaggg agccaattgt tccagttcat |
| 241 | ctatcacatg gccagttggt ccattcaaca aatggttatt ggatgcccat tatgtggcag |
| 301 | gcactgttcc gggggagagc acacaggtga actccagaaa gaagaagcta tggccgcagt |
| 361 | gattctggag agcatctttc tgaagcgatc ccaacagaaa aagaaaacat cacctctaaa |
| 421 | cttcaagaag cgcctgtttc tcttgaccgt gcacaaactc tcctactatg agtatgactt |
| 481 | tgaacgtggg agaagaggca gtaagaaggg ttcaatagat gttgagaaga tcacttgtgt |
| 541 | tgaaacagtg gttcctgaaa aaaatcctcc tccagaaaga cagattccga gaagaggtga |
| 601 | agagtccagt gaaatggagc aaatttcaat cattgaaagg ttcccttatc ccttccaggt |
| 661 | tgtatatgat gaagggcctc tctacgtctt ctccccaact gaagaactaa ggaagcggtg |
| 721 | gattcaccag ctcaaaaacg taatccggta caacagtgat ctggttcaga aatatcaccc |
| 781 | ttgcttctgg atcgatgggc agtatctctg ctgctctcag acagccaaaa atgctatggg |
| 841 | ctgccaaatt ttggagaaca ggaatggaag cttaaaacct gggagttctc accggaagac |
| 901 | aaaaaagcct cttcccccaa cgcctgagga ggaccagatc ttgaaaaagc cactaccgcc |
| 961 | tgagccagca gcagcaccag tctccacaag tgagctgaaa aaggttgtgg ccctttatga |
| 1021 | ttacatgcca atgaatgcaa atgatctaca gctgcggaag ggtgatgaat attttatctt |
| 1081 | ggaggaaagc aacttaccat ggtggagagc acgagataaa aatgggcagg aaggctacat |
| 1141 | tcctagtaac tatgtcactg aagcagaaga ctccatagaa atgtatgagt ggtattccaa |
| 1201 | acacatgact cggagtcagg ctgagcaact gctaaagcaa gaggggaaag aaggaggttt |
| 1261 | cattgtcaga gactccagca aagctggcaa atatacagtg tctgtgtttg ctaaatccac |
| 1321 | aggggaccct caaggggtga tacgtcatta tgttgtgtgt tccacacctc agagccagta |
| 1381 | ttacctggct gagaagcacc ttttcagcac catccctgag ctcattaact accatcagca |
| 1441 | caactctgca ggactcatat ccaggctcaa atatccagtg tctcaacaaa acaagaatgc |
| 1501 | accttccact gcaggcctgg gatacggatc atgggaaatt gatccaaagg acctgacctt |
| 1561 | cttgaaggag ctggggactg gacaatttgg ggtagtgaag tatgggaaat ggagaggcca |
| 1621 | gtacgacgtg gccatcaaga tgatcaaaga aggctccatg tctgaagatg aattcattga |
| 1681 | agaagccaaa gtcatgatga atctttccca tgagaagctg gtgcagttgt atggcgtctg |
| 1741 | caccaagcag cgccccatct tcatcatcac tgagtacatg gccaatggct gcctcctgaa |
| 1801 | ctacctgagg gagatgcgcc accgcttcca gactcagcag ctgctagaga tgtgcaagga |
| 1861 | tgtctgtgaa gccatggaat acctggagtc aaagcagttc cttcaccgag acctggcagc |
| 1921 | tcgaaactgt ttggtaaacg atcaaggagt tgttaaagta tctgatttcg gcctgtccag |
| 1981 | gtatgtcctg gatgatgaat acacaagctc agtaggctcc aaatttccag tccggtggtc |
| 2041 | cccaccggaa gtcctgatgt atagcaagtt cagcagcaaa tctgacattt gggcttttgg |
| 2101 | ggttttgatg tgggaaattt actccctggg gaagatgcca tatgagagat ttactaacag |
| 2161 | tgagactgct gaacacattg cccaaggcct acgtctctac aggcctcatc tggcttcaga |
| 2221 | gaaggtatat accatcatgt acagttgctg gcatgagaaa gcagatgagc gtcccacttt |
| 2281 | caaaattctt ctgagcaata ttctagatgt catggatgaa gaatcctgag ctcgccaata |
| 2341 | agcttcttgg ttctacttct cttctccaca agccccaatt tcactttctc agaggaaatc |
| 2401 | ccaagcttag gagccctgga gcctttgtgc tcccactcaa tacaaaaagg cccctctcta |
| 2461 | catctgggaa tgcacctctt ctttgattcc ctgggatagt ggcttctgag caaaggccaa |
| 2521 | gaaattattg tgcctgaaat ttcccgagag aattaagaca gactgaattt gcgatgaaaa |
| 2581 | tattttttag gagggaggat gtaaatagcc gcacaaaggg gtccaacagc tctttgagta |
| 2641 | ggcatttggt agagcttggg ggtgtgtgtg tgggggtgga ccgaatttgg caagaatgaa |
| 2701 | atggtgtcat aaagatggga ggggagggtg ttttgataaa ataaaattac tagaaagctt |
| 2761 | gaaa |

SEQ ID NO. 21

| | |
|---|---|
| 1 | MASWSIQQMV IGCPLCGRHC SGGEHTGELQ KEEAMAAVIL ESIFLKRSQQ |
| 51 | KKKTSPLNFK KRLFLLTVHK LSYYEYDFER GRRGSKKGSI DVEKITCVET |
| 101 | VVPEKNPPPE RQIPRRGEES SEMEQISIIE RFPYPFQVVY DEGPLYVFSP |
| 151 | TEELRKRWIH QLKNVIRYNS DLVQKYHPCF WIDGQYLCCS QTAKNAMGCQ |
| 201 | ILENRNGSLK PGSSHRKTKK PLPPTPEEDQ ILKKPLPPEP AAAPVSTSEL |
| 251 | KKVVALYDYM PMNANDLQLR KGDEYFILEE SNLPWWRARD KNGQEGYIPS |
| 301 | NYVTEAEDSI EMYEWYSKHM TRSQAEQLLK QEGKEGGFIV RDSSKAGKYT |
| 351 | VSVFAKSTGD PQGVIRHYVV CSTPQSQYYL AEKHLFSTIP ELINYHQHNS |
| 401 | AGLISRLKYP VSQQNKNAPS TAGLGYGSWE IDPKDLTFLK ELGTGQFGVV |
| 451 | KYGKWRGQYD VAIKMIKEGS MSEDEFIEEA KVMMNLSHEK LVQLYGVCTK |
| 501 | QRPIFIITEY MANGCLLNYL REMRHRFQTQ QLLEMCKDVC EAMEYLESKQ |
| 551 | FLHRDLAARN CLVNDQGVVK VSDFGLSRYV LDDEYTSSVG SKFPVRWSPP |
| 601 | EVLMYSKFSS KSDIWAFGVL MWEIYSLGKM PYERFTNSET AEHIAQGLRL |
| 651 | YRPHLASEKV YTIMYSCWHE KADERPTFKI LLSNILDVMD EES |

Unless otherwise defined, all technical and scientific terms used herein have the same meaning as commonly understood by one of ordinary skill in the art to which this invention belongs.

The embodiments illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that optional features, modification, improvement and variation of the embodiments herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1-3. (canceled)

4. A method of treating cancer in a subject in need thereof, the method comprising administering an inhibitor of palmitoylation of a ALB1 kinase.

5. The method of claim 4, where the inhibitor is selected from the group consisting of a nucleic acid, a small molecule, a peptide, a vector, and an antibody, wherein optionally said nucleic acid is selected from the group consisting of an siRNA, miRNA, an antisense nucleic acid, and an shRNA.

6. A method of treating cancer in a subject in need thereof, the method comprising administering an inhibitor of palmitoylation of a ALB2 kinase.

7. The method of claim 6, where the inhibitor is selected from the group consisting of a nucleic acid, a small molecule, a peptide, a vector, and an antibody, wherein optionally said nucleic acid is selected from the group consisting of an siRNA, miRNA, an antisense nucleic acid, and an shRNA.

8. A method of treating cancer in a subject in need thereof, the method comprising administering an inhibitor of palmitoylation of a PDPK1 kinase.

9. The method of claim 8, where the inhibitor is selected from the group consisting of a nucleic acid, a small molecule, a peptide, a vector, and an antibody, wherein optionally said nucleic acid is selected from the group consisting of an siRNA, miRNA, an antisense nucleic acid, and an shRNA.

10. The method of claim 5, wherein the antisense nucleic acid is selected from the group consisting of SEQ ID NO. 2, SEQ ID NO. 3, SEQ ID NO. 4, SEQ ID NO. 5, and SEQ ID NO. 6.

11. The method of claim 7, wherein the antisense nucleic acid is selected from the group consisting of SEQ ID NO. 8, SEQ ID NO. 9, SEQ ID NO. 10, SEQ ID NO. 11, and SEQ ID NO. 12.

12. The method of claim 9, wherein the antisense nucleic acid is selected from the group consisting of SEQ ID NO. 14, SEQ ID NO. 15, SEQ ID NO. 16, SEQ ID NO. 17, SEQ ID NO. 18, and SEQ ID NO. 19.

13-14. (canceled)
